(19) United States
(12) Patent Application Publication
(10) Pub. No.: US 2026/0069550 A1
(43) Pub. Date: Mar. 12, 2026

(54) TRANSDERMAL DELIVERY OF ANTIDIABETIC AGENTS

(71) Applicants: Christine RITTENHOUSE, Doylestown, PA (US); Nagaraj SRINIVASA, Glendale, CA (US); Arutyun Harry AVETISYAN, Glendale, CA (US); Frank JONES, Doylestown, PA (US)

(72) Inventors: Christine RITTENHOUSE, Doylestown, PA (US); Nagaraj SRINIVASA, Glendale, CA (US); Arutyun Harry AVETISYAN, Glendale, CA (US); Frank JONES, Doylestown, PA (US)

(21) Appl. No.: 19/321,013

(22) Filed: Sep. 5, 2025

Related U.S. Application Data
(60) Provisional application No. 63/691,712, filed on Sep. 6, 2024.

(51) Int. Cl. A61K 9/70; A61K 9/00; A61K 31/351; A61K 31/357; A61K 31/381; A61K 31/382; A61K 45/06

(52) U.S. Cl. CPC A61K 9/7061; A61K 9/0014; A61K 31/351; A61K 31/357; A61K 31/381; A61K 31/382; A61K 45/06

(57) ABSTRACT

Disclosed herein are compositions and methods for transdermal formulations of antidiabetic agents. An example transdermal formulation contains a SGLT2 inhibitor, and is used for the treatment of Type 2 diabetes.

### CROSS-REFERENCE TO RELATED APPLICATIONS

This application claims the benefit of priority to U.S. Provisional Application No. 63/691,712, filed Sep. 6, 2024, the contents of which is hereby incorporated by reference herein.

### BACKGROUND

Sodium-Glucose Linked Transporter 2 (SGLT2) inhibitors are a class of therapeutic agents used to treat Type 2 diabetes. FDA-approved SGLT2 inhibitors for treatment of Type 2 diabetes include canagliflozin, dapagliflozin, empagliflozin, and ertugliflozin, administered orally via tablets.

Transdermal administration of therapeutic agents is an alternative to oral administration, offering the advantages of minimally invasive drug delivery and potentially reduced side effects through bypassing metabolism in the liver.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the invention provides a pharmaceutical composition comprising a transdermal patch, the transdermal patch comprising a mixture of SGLT2 inhibitor and an acrylic adhesive.

In some embodiments, the invention provides a pharmaceutical composition comprising a transdermal patch, the transdermal patch comprising one and no more than one drug-in-adhesive layer, wherein the drug-in-adhesive layer comprises a mixture of a SGLT2 inhibitor and an adhesive.

In some embodiments, the invention provides a pharmaceutical composition comprising a transdermal patch, the transdermal patch comprising a SGLT2 inhibitor, wherein the transdermal patch has a surface area of from about 1 square centimeter to about 100 square centimeters.

In some embodiments, the invention provides a pharmaceutical composition comprising a transdermal patch, the transdermal patch comprising a SGLT2 inhibitor, wherein the transdermal patch has a thickness from about 0.1 millimeter to about 10 millimeters.

In some embodiments, the invention provides a pharmaceutical composition comprising a transdermal patch, the transdermal patch comprising an antidiabetic agent, wherein the transdermal patch has a mass of from about 10 milligrams to about 10 grams.

transdermal patch, the transdermal patch comprising an antidiabetic agent, wherein the transdermal patch exhibits a tack value of at least 5 grams, as determined by applying the transdermal patch to a stainless-steel cylinder, mechanically separating the transdermal patch from the stainless-steel cylinder, and measuring a level of effort required to separate the transdermal patch from the stainless-steel cylinder.

In some embodiments, the invention provides a pharmaceutical composition comprising a transdermal patch, the transdermal patch comprising an antidiabetic agent, wherein the patch exhibits a 90° peel adhesion of at least 50 grams, as determined by applying the transdermal patch to a steel plate, mechanically separating the transdermal patch from the steel plate by pulling the transdermal patch at a 90° angle, and measuring a level of effort required to separate the transdermal patch from the steel plate.

In some embodiments, the invention provides a pharmaceutical composition comprising a transdermal patch, the transdermal patch comprising an antidiabetic agent, wherein the patch exhibits a 180° peel adhesion of at least 50 grams, as determined by applying the transdermal patch to a steel plate, mechanically separating the transdermal patch from the steel plate by pulling the transdermal patch at a 180° angle, and measuring a level of effort required to separate the transdermal patch from the steel plate.

In some embodiments, the invention provides a pharmaceutical composition comprising a transdermal patch, the transdermal patch comprising a SGLT2 inhibitor, wherein if in a study the transdermal patch is applied to cadaver skin and permeation of the SGLT2 inhibitor across the cadaver skin is monitored, then a permeation profile of at least 7 days is observed.

In some embodiments, the invention provides a pharmaceutical composition comprising a transdermal patch, the transdermal patch comprising a SGLT2 inhibitor, wherein,
if in a study:
a) the transdermal patch is placed into a first hydrochloric acid solution at an acidic pH;
b) a profile at which the SGLT2 inhibitor migrates into the first hydrochloric acid solution over a period of 7 days is determined;
c) an oral form of the SGLT2 inhibitor is placed into a second hydrochloric acid solution at the acidic pH; and
d) a profile at which the SGLT2 inhibitor from the oral form migrates into the second hydrochloric acid solution over a period of 7 days is determined,
then the profile at which the SGLT2 inhibitor migrates into the first solution from the transdermal patch shows a total release at 7 days of less than a total release observed in the profile at which the SGLT2 inhibitor from the oral form migrates into the second solution within 12 hours.

In some embodiments, the invention provides a pharmaceutical composition comprising a transdermal patch, the transdermal patch comprising a SGLT2 inhibitor, wherein,
if in a study:
a) the transdermal patch is placed into a sodium phosphate solution at a basic pH; and
b) a profile at which the SGLT2 inhibitor migrates into the solution is determined, then the profile at which the SGLT2 inhibitor migrates into the solution from the transdermal patch shows a total release of SGLT2 inhibitor over a period of at least about least 7 days.

In some embodiments, the invention provides a pharmaceutical composition comprising a mixture of a SGLT2 inhibitor and an acrylic adhesive.

In some embodiments, the invention provides a method of treating a condition, the method comprising administering to a subject in need thereof a transdermal patch, the transdermal patch comprising a SGLT2 inhibitor, wherein the transdermal patch delivers the SGLT2 inhibitor over a period of at least about 7 days.

In some embodiments, the invention provides a method of treating a condition, the method comprising administering to a subject in need thereof a therapeutically-effective amount of an antidiabetic agent via a transdermal patch, the transdermal patch comprising the antidiabetic agent, wherein the transdermal patch has a mass from about 10 milligrams to about 10 grams.

In some embodiments, the invention provides a method of treating Type 2 diabetes, the method comprising applying a transdermal patch to a subject in need thereof, wherein the transdermal patch comprises a therapeutically-effective amount of an antidiabetic agent, wherein the transdermal patch is applied to a skin surface of a subject.

In some embodiments, the invention provides a method comprising:
a) contacting a skin surface of a subject in need thereof with a first sample of a mixture, the mixture comprising a therapeutically-effective amount of an antidiabetic agent and an adhesive; and
b) 7 days after the contacting, contacting the subject with a second sample of the mixture.

In some embodiments, the invention provides a method of treating Type 2 diabetes, the method comprising contacting a skin surface of a subject in need thereof with a mixture, the mixture comprising a therapeutically-effective amount of an antidiabetic agent and an adhesive, wherein the antidiabetic agent has a molecular weight of less than about 1000 Daltons.

In some embodiments, the invention provides a method comprising administering to a subject in need thereof a transdermal patch, the transdermal patch comprising a mixture of a therapeutically-effective amount of an antidiabetic agent and an adhesive, wherein if in a study the transdermal patch is applied to a study subject, then the study subject exhibits a Tmax of the antidiabetic agent at least 5 hours after the transdermal patch is applied to the study subject.

In some embodiments, the invention provides a method comprising administering to a subject a transdermal patch, wherein the transdermal patch comprises a SGLT2 inhibitor, wherein the transdermal patch delivers to the subject at least 50% of the SGLT2 inhibitor in the transdermal patch.

In some embodiments, the invention provides a method of preparing a formulation, the method comprising:
a. contacting a solvent with a SGLT2 inhibitor to provide a first mixture;
b. contacting the first mixture with a permeation enhancer to provide a second mixture; and
c. contacting the second mixture with an acrylic adhesive to provide the formulation.

In some embodiments, the invention provides a method of preparing a formulation, the method comprising:
a. contacting a solvent with a SGLT2 inhibitor to provide a first mixture; and
b. contacting the first mixture with an acrylic adhesive to provide the formulation.

In some embodiments, the invention provides a method of preparing a transdermal patch, the method comprising:
a. combining a SGLT2 inhibitor, an adhesive, and a solvent to provide a crude mixture; and
b. preparing a uniform mixture by stirring the crude mixture until the crude mixture becomes substantially uniform.

In some embodiments, the invention provides a method comprising applying a mixture to a release liner, wherein the mixture comprises a SGLT2 inhibitor and an adhesive, and the release liner is a polyester film.

In some embodiments, the invention provides a method comprising applying a transdermal patch to a subject, the transdermal patch comprising:
a. a release liner; and
b. a mixture of SGLT2 inhibitor and an adhesive.

In some embodiments, the invention provides a method comprising:
a. inserting a transdermal patch into an aluminum packaging material to provide a pouch, wherein the transdermal patch comprises an antidiabetic agent; and
b. sealing the pouch.

In some embodiments, the invention provides an article of manufacture comprising:
a. a transdermal patch comprising an antidiabetic agent;
b. aluminum packaging material; and
c. written instructions on use of the transdermal patch to treat diabetes.

The present invention also provides for a transdermal patch containing: at least one SGLT2 inhibitor or antidiabetic agent; an adhesive layer containing an acrylic adhesive in which the SGLT2 inhibitor and/or or antidiabetic agent is incorporated; optionally, one or more permeation enhancers in the adhesive layer, to facilitate delivery of the SGLT2 inhibitor and/or or antidiabetic agent through the skin; a removeable release liner, positioned on one surface of the adhesive layer, configured to be removed prior to application; and a backing film on the opposed surface of the adhesive layer, impermeable or occlusive to protect the patch and control drug release direction; wherein the adhesive layer is positioned between the removable release liner and the backing film, such that upon removal of the liner and placement on a skin surface, drug permeation is directed outward from the adhesive-contacting layer toward the skin.

The present invention also provides for a kit containing: a transdermal patch described herein; an aluminum packaging material enclosing the transdermal patch; and written instructions for use of the transdermal patch to treat diabetes.

The present invention also provides for a method of treating diabetes in a subject in need thereof, the method including: administering to the subject a transdermal patch, the transdermal patch including a mixture of a SGLT2 inhibitor and an acrylic adhesive, wherein the transdermal patch is applied to a skin surface of the subject in an amount effective to treat diabetes.

### BRIEF DESCRIPTION OF THE FIGURES

FIG. 1 provides the dapagliflozin delivery (mg) from formulations 39B, 39C, and 39D over 172 hours from skin permeation studies described in Example 3, plotted as delivery every 24 hours.

FIG. 2 provides the dapagliflozin delivery (mg) from formulations 39B, 39C, and 39D over 172 hours from skin permeation studies described in Example 3, plotted as cumulative delivery.

FIG. 3 provides the dapagliflozin delivery (mg) from formulation 39B over 172 hours from skin permeation studies described in Example 3, plotted as delivery every 24 hours.

FIG. 4 provides the dapagliflozin delivery (mg) from formulation 39B over 172 hours from skin permeation studies described in Example 3, plotted as cumulative delivery.

FIG. 5 provides the dapagliflozin delivery (mg) from formulation 39C over 172 hours from skin permeation studies described in Example 3, plotted as delivery every 24 hours.

FIG. 6 provides the dapagliflozin delivery (mg) from formulation 39C over 172 hours from skin permeation studies described in Example 3, plotted as cumulative delivery.

FIG. 7 provides the dapagliflozin delivery (mg) from formulation 39D over 172 hours from skin permeation studies described in Example 3, plotted as delivery every 24 hours.

FIG. 8 provides the dapagliflozin delivery (mg) from formulation 39D over 172 hours from skin permeation studies described in Example 3, plotted as cumulative delivery.

FIG. 9 provides the dapagliflozin release (mg) from formulation 51A over 172 hours from dissolution studies described in Example 5.

FIG. 10 compares the dapagliflozin release (mg) from formulation 51A and the oral form of dapagliflozin over 172 hours from dissolution studies described in Example 5.

FIG. 11 provides the dapagliflozin release (mg) from the oral form of dapagliflozin over 1.3 hours from dissolution studies described in Example 5.

FIG. 12 provides the HPLC trace of dapagliflozin.

FIG. 13 provides images of subjects with a placebo transdermal patch upon initial application and removal of the patch after 24 hours.

FIG. 14 illustrates a monolith-style path design with a removable release liner, a drug in adhesive and/or enhancer mixture, and a backing film.

FIG. 15 provides a top view of the hand drawdown coater.

FIG. 16 provides a side view of the pilot coating machine.

FIG. 17 shows a side view of the commercial coating machine.

FIG. 18 shows where the score line is cut in the release liner.

### DETAILED DESCRIPTION

### Sodium-Glucose Cotransporters

Glucose is an abundant monosaccharide and crucial energy source for living cells.

Glucose transport across cell membranes is mediated by two main types of transporters: glucose transporters (GLUTs) that transport glucose through diffusion and sodium-glucose cotransporters (SGLTs) that transport glucose against the concentration gradient by coupling to sodium. Glucose transporters are involved in many metabolic processes. Impaired glucose transport is a significant contributor to diabetes development and progression; as such, modulators of glucose transporters are important therapeutics for maintaining glucose homeostasis.

SGLTs have become an important target for diabetes treatment. The main isoforms of SGLT are SGLT1 and SGLT2, the latter of which is responsible for roughly 90% of the kidney's glucose reabsorption. Inhibition of SGLT2 blocks glucose reabsorption and promotes elimination of glucose through the urine, lowering blood glucose levels.

### SGLT2 Inhibitors

SGLT2 inhibitors, known as gliflozins, are a class of drugs that modulate reabsorption of glucose in the kidneys. These inhibitors reduce glucose reabsorption by the kidneys and induce excretion of glucose through the urine to lower blood glucose independent of insulin activity.

Oral SGLT2 inhibitors are absorbed into the bloodstream, bind to plasma proteins, and are eventually filtered from the plasma at the glomerulus. In the glomerulus, SGLT2 inhibitors bind to SGLT2 in the early segments of the proximal tubule, where the inhibitors can block glucose reabsorption. Glucose that is not blocked in the early segments of the proximal tubule is reabsorbed by SGLT1 in the late proximal tubule.

SGLT2 inhibitors are C or O-aryl glucosides. SGLT2 inhibitors are proposed to bind to the extracellular surface of SGLT2, with the glucose moiety of the inhibitor binding to the glucose binding site of SGLT2 and the aglycone moiety of the inhibitor binding to a site adjacent to the glucose binding site. Once bound, the SGLT2 inhibitors force SGLT2 in an outward facing conformation, blocking glucose reabsorption. Binding of SGLT2 inhibitors to the glucose binding site is reversible but is slow due to additional interactions between the aglycone and the site adjacent to the glucose binding site. SGLT2 inhibitors also have off-target effects on cardiac sodium proton exchangers (NHE1), leading to a lower progression of cardiac disease.

In some embodiments, a SGLT2 inhibitor in a pharmaceutical composition described herein is canagliflozin, dapagliflozin, empagliflozin, ertugliflozin, ipragliflozin, luseogliflozin, tofogliflozin, or a solvate or hydrate thereof. In some embodiments, the SGLT2 inhibitor is canagliflozin, dapagliflozin, empagliflozin, or ertugliflozin. In some embodiments, the SGLT2 inhibitor is dapagliflozin. In some embodiments, the SGLT2 inhibitor is dapagliflozin propane diol hydrate.

### Pharmaceutical Compositions

The present disclosure provides pharmaceutical compositions comprising one or more antidiabetic agents. In some embodiments, the antidiabetic agent is an alpha-glucosidase inhibitor, amylin analogue, dipeptidyl peptidase 4 (DPP-4) inhibitor, incretin mimetic, metglinide, insulin, non-sulfonylurea, SGLT2 inhibitor, sulfonylurea, or thiazolidinedione. In some embodiments, the antidiabetic agent is acarbose, miglitol, pramlintide, alogliptan, linagliptan, saxagliptin, sitagliptin, albiglutide, dulaglutide, exenatide, liraglutide, lixisenatide, nateglinide, repaglinide, metformin, canagliflozin, dapagliflozin, empagliflozin, ertugliflozin, chlorpropamide, glimepiride, glipizide, glyburide, tolazamide, tolbutamide, rosiglitazone, or pioglitazone. In some embodiments, the antidiabetic agent is a SGLT2 inhibitor. In some embodiments, the antidiabetic agent is canagliflozin, dapagliflozin, empagliflozin, ertugliflozin or a solvate or hydrate thereof. In some embodiments, the antidiabetic agent is dapagliflozin. In some embodiments, the antidiabetic agent is dapagliflozin propane diol hydrate.

In some embodiments, the antidiabetic agent is present in a therapeutically-effective amount for the treatment of a condition. In some embodiments, the antidiabetic agent is present in a therapeutically-effective amount for the treatment of diabetes. In some embodiments, the antidiabetic agent is present in a therapeutically-effective amount for the treatment of Type 2 diabetes.

In some embodiments, the SGLT2 inhibitor is present in a therapeutically-effective amount for the treatment of a condition. In some embodiments, the SGLT2 inhibitor is present in a therapeutically-effective amount for the treatment of diabetes. In some embodiments, the SGLT2 inhibitor is present in a therapeutically-effective amount for the treatment of Type 2 diabetes.

The present disclosure provides pharmaceutical compositions comprising one or more adhesives. In some embodiments, the adhesive is a pressure-sensitive adhesive. In some embodiments, the adhesive is an acrylic adhesive, silicone adhesive, or polyisobutene adhesive. In some embodiments, the adhesive is an acrylic adhesive. In some embodiments, the adhesive is an acrylate polymer. In some embodiments, the acrylate polymer comprises one or more of acrylate, methacrylate, methyl methacrylate, acrylic acid, or vinyl acetate monomers. In some embodiments, the adhesive is crosslinked. In some embodiments, the adhesive is functionalized with crosslinking groups, for example, groups comprising acids or alcohols.

Non-limiting examples of adhesives include: GELVA® GMS 788, DURO-TAK® 87-2051, DURO-TAK® 87-2051, DURO-TAK® 87-2052, DURO-TAK® 87-2074, DURO-TAK® 87-2516, DURO-TAK® 87-2852, DURO-TAK® 87-4287, Silbione® RT GEL 4717A and 4717B, OPPANOL® B12, DURO-TAK® 87-6908, Liveo™ BIO-PSA 7-4502, Liveo™ BIO-PSA 7-4602, and Liveo™ BIO-PSA 7-4301.

The present disclosure provides pharmaceutical compositions comprising one or more permeation enhancers. In some embodiments, the permeation enhancer is an organic acid, alcohol, surfactant, ether, fatty acid, fatty acid ester, sulfoxide, terpene, alkanone, povidone, copovidone, crosspovidone, alkyl sulfate, poloxamer, polysorbate, polyacrylate, or polyester.

Non-limiting examples of permeation enhancers include salicylic acid, citric acid, succinic acid, glycerol, propylene glycol, polyethylene glycol, diethylene glycol monoethyl ether, polyethylene glycol monolaurate, isopropyl myristate, isopropyl palmitate, ethyl oleate, oleyl oleate, sodium dodecyl sulfate, sodium laurate, sodium lauryl sulfate, limonene, polysorbate 20, polysorbate 80, castor oil, levulinic acid, lactic acid, eucalyptus oil, urea, polymethacrylate, polyvinylpyrrolidone, Kollidon® VA 64, Plasdone™ S-630, Plasdone™ C-30, Polyplasdone™ XL, Prosolv® 730, Plastoid® B, Kollidon® CL, povidone K90, povidone K-17, and povidone K29/32. In some embodiments, the permeation enhancer is an organic acid. In some embodiments, the permeation enhancer is a keto acid. In some embodiments, the permeation enhancer is a nonionic surfactant. In some embodiments, the permeation enhancer is levulinic acid. In some embodiments, the permeation enhancer is polysorbate 80.

The present disclosure provides pharmaceutical compositions comprising one or more solvents. In some embodiments, the solvent is an organic solvent. In some embodiments, the solvent is an aqueous solvent. In some embodiments, the solvent is an alcohol. Non-limiting examples of alcohols include C₁₋₆ alkyl linear alcohols, C₁₋₆ alkyl branched alcohols, alkylene glycols, and polyalkylene glycols. In some embodiments, the solvent is methanol. In some embodiments, the solvent is heptane. In some embodiments, the solvent is ethyl acetate. In some embodiments, the solvent is dimethylsulfoxide. In some embodiments, the solvent is water.

In some embodiments, a pharmaceutical composition described herein is a mixture of an antidiabetic agent and an adhesive. In some embodiments, a pharmaceutical composition described herein is a mixture of an antidiabetic agent, adhesive, and a permeation enhancer. In some embodiments, a pharmaceutical composition described herein is a mixture of an antidiabetic agent and an acrylic adhesive. In some embodiments, a pharmaceutical composition described herein is a mixture of an antidiabetic agent, an acrylic adhesive, and a permeation enhancer.

In some embodiments, a pharmaceutical composition described herein is a mixture of a SGLT2 inhibitor and an adhesive. In some embodiments, a pharmaceutical composition described herein is a mixture of a SGLT2 inhibitor, adhesive, and a permeation enhancer. In some embodiments, a pharmaceutical composition described herein is a mixture of a SGLT2 inhibitor and an acrylic adhesive. In some embodiments, a pharmaceutical composition described herein is a mixture of a SGLT2 inhibitor, an acrylic adhesive, and a permeation enhancer.

In some embodiments, a pharmaceutical composition described herein is a mixture of dapagliflozin and an adhesive. In some embodiments, a pharmaceutical composition described herein is a mixture of dapagliflozin, adhesive, and a permeation enhancer. In some embodiments, a pharmaceutical composition described herein is a mixture of dapagliflozin and an acrylic adhesive. In some embodiments, a pharmaceutical composition described herein is a mixture of a dapagliflozin, an acrylic adhesive, and a permeation enhancer.

A pharmaceutical composition described herein can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism.

In some embodiments, an antidiabetic agent in a pharmaceutical composition disclosed herein is present in an amount from about 5% to about 50% of the mass of the pharmaceutical composition on an all-solids basis. In some embodiments, the antidiabetic agent is present in an amount from about 5% to about 10%, about 5% to about 15%, about 5% to about 20%, about 5% to about 25%, about 5% to about 30%, about 5% to about 35%, about 5% to about 40%, or about 5% to about 45% of the mass of the pharmaceutical composition on an all-solids basis. In some embodiments, the antidiabetic agent is present in an amount of about 5%, about 10%, about 15%, about 20%, about 25%, about 30%, about 35%, about 40%, about 45%, or about 50% of the mass of the pharmaceutical composition on an all-solids basis. In some embodiments, the antidiabetic agent is present in amount of about 15%, about 16%, about 17%, about 18%, about 19%, about 20%, about 21%, about 22%, about 23%, about 24%, or about 25% of the mass of the pharmaceutical composition on an all-solids basis.

In some embodiments, a component in a pharmaceutical composition disclosed herein is present as a solid. In some embodiments, a component in a pharmaceutical composition disclosed herein is present as a liquid. In some embodiments, a component in a pharmaceutical composition disclosed herein is present as solid percentage, or solids basis, calculated as the percentage of a mass of a solid component divided by the total mass of the solid suspended, in solution or dispersed in a liquid component. In some embodiments, the component has a solids basis is about 20% to about 99.99%. In some embodiments, the component has a solids basis is about 20% to about 30%, about 20% to about 40%, about 20% to about 50%, about 20% to about 60%, about 20% to about 70%, about 20% to about 80%, about 20% to about 90%, or about 20% to about 99.99%. In some embodiments, the component solids basis is about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, about 20% to about 85%, about 20% to about 90%, about 20% to about 95%, about 20% to about 99.99%, about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 40% to about 99.99%, about 60% to about 65%, about 60% to about 70%, about 60% to about 75%, about 60% to about 80%, about 60% to about 85%, about 60% to about 90%, about 60% to about 95%, or about 60% to about 99.99%.

In some embodiments, the component has a solids basis is about 20%, about 30%, about 40%, about 50%, about 60%, about 70%, about 80%, about 90%, or about 99.99%. In some embodiments, solids basis is about 20%, about 25%, about 30%, about 35%, about 40%, about 45%, about 50%, about 55%, about 60%, about 65%, about 70%, about 75%, about 80%, about 85%, about 90%, about 95%, or about 99.99%. In some embodiments, the component has a solids basis is about 35%, about 35.5%, about 36%, about 36.5%, about 37%, about 37.5%, about 38%, about 38.5%, about 39%, about 39.5%, about 40%, about 40.5%, about 41%, about 41.5%, about 42%, about 42.5%, about 43%, about 43.5%, about 44%, about 44.5%, about 45%, about 45.5%, about 46%, about 46.5%, about 47%, about 47.5%, about 48%, about 48.5%, about 49%, about 49.5%, or about 50%. In some embodiments, the component has a solids basis is about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.5%, or about 99.99%. In some embodiments, the component has a solids basis is about 41.5%. In some embodiments, the component has a solids basis is about 95%. In some embodiments, the component has a solids basis is about 98%.

In some embodiments, a component in a pharmaceutical composition disclosed herein can be at least can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

In some embodiments, the pharmaceutical composition can be administered to a subject for a period of at least about 1 day to about 1 year. In some embodiments, the pharmaceutical composition can be administered to a subject for about 1 day to about 7 days, about 1 day to about 14 days, about 1 day to about 21 days, about 1 day to about 28 days, about 1 day to about 1 month, about 1 day to about 2 months, about 1 day to about 3 months, about 1 day to about 6 months, about 1 day to about 1 year. In some embodiments, the pharmaceutical composition can be administered to a subject for about 1 day, about 7 days, about 14 days, about 21 days, about 28 days, about 1 month, about 2 months, about 3 months, about 6 months, or about 1 year. In some embodiments, the pharmaceutical composition can be administered to a subject for about 1 days. In some embodiments, the pharmaceutical composition can be administered to a subject for about 7 days.

In some embodiments, the pharmaceutical composition is reapplied to the subject after initial application. In some embodiments, the pharmaceutical composition is reapplied about 1 day to about 1 month after initial application. In some embodiments, the pharmaceutical is reapplied about 1 day to about 7 days, about 1 day to about 14 days, about 1 day to about 21 days, about 1 day to about 28 days, or about 1 day to about 1 month after initial application. In some embodiments, the pharmaceutical composition is reapplied about 1 day, about 7 days, about 14 days, about 21 days, about 28 days, or about 1 month after initial application. In some embodiments, the pharmaceutical composition is reapplied about 1 day after initial application. In some embodiments, the pharmaceutical composition is reapplied about 7 days after initial application.

In some embodiments, the pharmaceutical composition can be administered to a skin surface of a subject. In some embodiments, the skin surface comprises an arm, shoulder, wrist, leg, chest, abdomen, or face of a subject.

The present disclosure provides a method of preparation a formulation, the method comprising:
a. contacting a solvent with a SGLT2 inhibitor to provide a first mixture;
b. contacting the first mixture with a permeation enhancer to provide a second mixture; and
c. contacting the second mixture with an acrylic adhesive to provide the formulation.

In some embodiments, the formulation is prepared by
a. contacting a solvent with a SGLT2 inhibitor to provide a first mixture; and
b. contacting the first mixture with an acrylic adhesive to provide a formulation.

### Transdermal Systems

The present disclosure provides pharmaceutical compositions suitable for use in transdermal systems. In some embodiments, the pharmaceutical compositions are applied as a topical composition, such as solutions, suspensions, lotions, gels, pastes, medicated sticks, balms, creams, and ointments. In some embodiments, the pharmaceutical compositions are applied as a transdermal device, for example, a transdermal patch. In some embodiments, the transdermal device is a drug-in-adhesive patch, a reservoir patch, or a matrix patch. In some embodiments, the transdermal device is a drug-in-adhesive patch.

In some embodiments, the transdermal device comprises a backing film, at least one drug-in-adhesive layer, a membrane layer, and a release liner. In some embodiments, the transdermal device comprises a backing film, at least one drug-in-adhesive layer, and a release liner.

In some embodiments, the drug-in-adhesive layer is about 1 to about 2, about 1 to about 3, about 1 to about 4, or about 1 to about 5 layers.

In some embodiments, the drug-in-adhesive layer is about 1, about 2, about 3, about 4, or about 5 layers. In some embodiments, the drug-in-adhesive layer is about 2 layers. In some embodiments, the drug-in-adhesive layer is about 1 layer.

In some embodiments, the release liner is a paper or a film. In some embodiments, the release liner is a polymer film. In some embodiments, the release liner is a hydrophobic polymer film. In some embodiments, the release liner is a polyester, polyethylene, polypropylene, polyurethane, or polystyrene film. In some embodiments, the release liner is a polyester film. In some embodiments, the polymer film is coated. In some embodiments, the polymer film is coated with a fluoropolymer or a silicone. In some embodiments, the release liner is a fluoropolymer-coated polyester, polyethylene, or polypropylene film. In some embodiments, the release liner is a fluoropolymer-coated polyester film. Non-limiting examples of release liners include 3M Scotchpak™ 9744, Loparex 4400, and 3M Scotchpak™ 1022. In some embodiments, the release liner is removed prior to applying the pharmaceutical composition to a subject.

In some embodiments, the backing film is a polymer film. In some embodiments, the backing film is a hydrophobic polymer. In some embodiments, the backing film is a polyester, polyethylene, polyurethane, polypropylene, polyvinyl chloride, polyethylene vinyl acetate, or polyethylene terephthalate film. In some embodiments, the backing film is a polyethylene film. In some embodiments, the polymer film is coated. In some embodiments, the polymer film is coated with a fluoropolymer or silicone. In some embodiments, the backing film is a fluoropolymer-coated polyester film. Non-limiting examples of backing films include 3M Scotchpak™ 1109, 3M Scotchpak™ 9720, and 3M Scotchpak™ 9738. In some embodiments, the backing film conforms to the area of administration on a subject. In some embodiments, the backing film is occlusive to an area of skin of the subject. In some embodiments, the backing film is impermeable to water.

### Preparation of Transdermal Systems

The present disclosure provides pharmaceutical compositions suitable for use in a transdermal patch.

The present disclosure provides a method of preparing a transdermal patch, the method comprising:
a. combining an antidiabetic agent, an adhesive, and a solvent to provide a crude mixture; and
b. preparing a uniform mixture by stirring the crude mixture until the crude mixture becomes substantially uniform.

In some embodiments, the crude mixture is stirred for about 5 minutes to about 3 hours. In some embodiments, the crude mixture is stirred for about 5 minutes to about 2.5 hours, about 5 minutes to about 2 hours, about 5 minutes to about 1.5 hours, about 5 minutes to about 1 hour, about 5 minutes to about 30 minutes, or about 5 minutes to about 10 minutes. In some embodiments, the crude mixture is stirred for about 5 minutes, about 10 minutes, about 30 minutes, about 1 hour, about 1.5 hours, about 2 hours, about 2.5 hours, or about 3 hours. In some embodiments, the crude mixture is stirred for about 30 minutes.

In some embodiments, the uniform mixture is applied to a release liner to provide a coated release liner. In some embodiments, the coated release liner is dried to provide a film. In some embodiments, the drying comprises a first stage and a second stage. In some embodiments, the first stage drying temperature is from about 50° C. to about 100° C. In some embodiments, the first stage drying temperature is about 50° C., about 60° C., about 70° C., about 80° C., about 90° C., or about 100° C. In some embodiments, the second stage drying temperature is from about 100° C. to about 150° C. In some embodiments, the second stage drying temperature is about 100° C., about 110° C., about 120° C., about 130° C., about 140° C., or about 150° C. In some embodiments, the dried film is nipped to a backing layer.

In some embodiments, the transdermal patch has a surface area of about 1 cm² to about 5 cm², about 1 cm² to about 10 cm², about 1 cm² to about 15 cm², about 1 cm² to about 20 cm², about 1 cm² to about 25 cm², about 1 cm² to about 30 cm², about 1 cm² to about 35 cm², about 1 cm² to about 40 cm², about 1 cm² to about 45 cm², about 1 cm² to about 50 cm², about 1 cm² to about 55 cm², about 1 cm² to about 60 cm², about 1 cm² to about 65 cm², about 1 cm² to about 70 cm², about 1 cm² to about 75 cm², about 1 cm² to about 80 cm², about 1 cm² to about 85 cm², about 1 cm² to about 90 cm², about 1 cm² to about 95 cm², or about 1 cm² to about 100 cm², about 5 cm² to about 10 cm², about 5 cm² to about 15 cm², about 5 cm² to about 20 cm², about 5 cm² to about 25 cm², about 5 cm² to about 30 cm², about 5 cm² to about 35 cm², about 5 cm² to about 40 cm², about 5 cm² to about 45 cm², about 5 cm² to about 50 cm², about 5 cm² to about 55 cm², about 5 cm² to about 60 cm², about 5 cm² to about 65 cm², about 5 cm² to about 70 cm², about 5 cm² to about 75 cm², about 5 cm² to about 80 cm², about 5 cm² to about 85 cm², about 5 cm² to about 90 cm², about 5 cm² to about 95 cm², or about 5 cm² to about 100 cm², about 25 cm² to about 30 cm², about 25 cm² to about 35 cm², about 25 cm² to about 40 cm², about 25 cm² to about 45 cm², about 25 cm² to about 50 cm², about 25 cm² to about 55 cm², about 25 cm² to about 60 cm², about 25 cm² to about 65 cm², about 25 cm² to about 70 cm², about 25 cm² to about 75 cm², about 25 cm² to about 80 cm², about 25 cm² to about 85 cm², about 25 cm² to about 90 cm², about 25 cm² to about 95 cm², or about 25 cm² to about 100 cm².

In some embodiments, the transdermal patch has a surface area of about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², or about 100 cm². In some embodiments, the transdermal patch has a surface area of about 25 cm². In some embodiments, the transdermal patch has a surface area of about 27.5 cm². In some embodiments, the transdermal patch has a surface area of about 30 cm². In some embodiments, the transdermal patch has a surface area of about 50 cm². In some embodiments, the transdermal patch has a surface area of about 75 cm². In some embodiments, the transdermal patch has a surface area of about 100 cm².

In some embodiments, the transdermal patch has a thickness of about 0.1 mm to about 10 mm. In some embodiments, the transdermal patch has a thickness of about 0.1 mm to about 0.2 mm, about 0.1 mm to about 0.3 mm, about 0.1 mm to about 0.4 mm, about 0.1 mm to about 0.5 mm, about 0.1 mm to about 0.6 mm, about 0.1 mm to about 0.7 mm, about 0.1 mm to about 0.8 mm, about 0.1 mm to about 0.9 mm, about 0.1 mm to about 1 mm, about 1 mm to about 5 mm, about 2 mm to about 5 mm, about 3 mm to about 5 mm, about 4 mm to about 5 mm, about 1 mm to about 10 mm, about 2 mm to about 10 mm, about 3 mm to about 10 mm, about 4 mm to about 10 mm, about 5 mm to about 10 mm, about 6 mm to about 10 mm, about 7 mm to about 10 mm, about 8 mm to about 10 mm, or about 9 mm to about 10 mm.

In some embodiments, the transdermal patch has a thickness of about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 2 mm, about 3 mm, about 4 mm, about 5 mm, about 6 mm, about 7 mm, about 8 mm, about 9 mm, or about 10 mm. In some embodiments, the transdermal patch has a thickness of about 0.1 mm, about 0.11 mm, about 0.12 mm, about 0.13 mm, about 0.14 mm, about 0.15 mm, about 0.16 mm, about 0.17 mm, about 0.18 mm, about 0.19 mm, about 0.20 mm, about 0.21 mm, about 0.22 mm, about 0.23 mm, about 0.24 mm, about 0.25 mm, about 0.26 mm, about 0.27 mm, about 0.28 mm, about 0.29 mm, about 0.30 mm, about 0.31 mm, about 0.32 mm, about 0.33 mm, about 0.34 mm, about 0.35 mm, about 0.36 mm, about 0.37 mm, about 0.37 mm, about 0.38 mm, about 0.39 mm, or about 0.40 mm. In some embodiments, the transdermal patch has a thickness of about 0.20 mm. In some embodiments, the transdermal patch has a thickness of about 0.21 mm. In some embodiments, the transdermal patch has a thickness of about 0.22 mm. In some embodiments, the transdermal patch has a thickness of about 0.23 mm. In some embodiments, the transdermal patch has a thickness of about 0.24 mm. In some embodiments, the transdermal patch has a thickness of about 0.25 mm. In some embodiments, the transdermal patch has a thickness of about 30 mm.

In some embodiments, the transdermal patch has a mass of about 0.01 g to about 10 g. In some embodiments, the transdermal patch has a mass of about 0.01 g to about 0.1 g, about 0.01 g to about 1 g, about 0.01 g to about 2 g, about 0.01 g to about 3 g, about 0.01 g to about 4 g, about 0.01 g to about 5 g, about 0.01 g to about 6 g, about 0.01 g to about 7 g, about 0.01 g to about 8 g, about 0.01 g to about 9 g, about 0.01 g to about 10 g, about 0.1 g to about 1 g, about 0.1 g to about 2 g, about 0.1 g to about 3 g, about 0.1 g to about 4 g, about 0.1 g to about 5 g, about 0.1 g to about 6 g, about 0.1 g to about 7 g, about 0.1 g to about 8 g, about 0.1 g to about 9 g, about 0.1 g to about 10 g, about 1 g to about 2 g, about 1 g to about 3 g, about 1 g to about 4 g, about 1 g to about 5 g, about 1 g to about 6 g, about 1 g to about 7 g, about 1 g to about 8 g, about 1 g to about 9 g, or about 1 g to about 10 g.

In some embodiments, the transdermal patch has a mass of about 0.01 g, about 0.1 g, about 0.2 g, about 0.3 g, about 0.4 g, about 0.5 g, about 0.6 g, about 0.7 g, about 0.8 g, about 0.9 g, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, or about 10 g. In some embodiments, the patch has a mass of about 0.2 g. In some embodiments, the patch has a mass of about 0.4 g. In some embodiments, the patch has a mass of about 0.6 g. In some embodiments, the patch has a mass of about 0.8 g. In some embodiments, the patch has a mass of about 1 g.

In some embodiments, the transdermal patch delivers to a subject from about 30% to about 99.99% of the antidiabetic agent in the transdermal patch. In some embodiments, the transdermal patch delivers to a subject from about 30% to about 40%, about 30% to about 50%, about 30% to about 60%, about 30% to about 70%, about 30% to about 80%, about 30% to about 90%, or about 30% to about 99% of the antidiabetic agent in the transdermal patch. In some embodiments, the transdermal patch delivers to a subject about 30%, about 40%, about 50%, about 60%, about 70%, about 80%, about 90%, about 99%, or about 99.99% of the antidiabetic agent in the transdermal patch. In some embodiments, the transdermal patch delivers to a subject about 50% of the antidiabetic agent in the transdermal patch.

### Evaluation of Transdermal Properties

Experimental methods used to evaluate transdermal properties include skin permeation, tack testing, shear testing, peel testing, and dissolution testing. A representative procedure for skin permeation testing is included in EXAMPLE 3. Representative procedures for tack, shear, and peel testing are included in EXAMPLE 4. A representative procedure for dissolution testing is included in EXAMPLE 5. A brief summary of the experimental methods used herein is described below.

Skin permeation: Permeation of a therapeutic agent across human skin is measured using a Franz diffusion cell. The diffusion cell has an upper donor compartment and a lower receptor compartment, between which a skin membrane is located. The Franz cell is surrounded by a water jacket to ensure a constant temperature. In this experiment, the upper donor compartment contains a transdermal patch with an antidiabetic agent, the skin membrane is human cadaver skin, and the lower receptor compartment contains a buffer solution that is mixed by a stir bar. Aliquots of the buffer solution are sampled to measure permeation of the antidiabetic agent across the skin membrane.

Tack testing: Tack determines how strongly an adhesive bond is formed. Tack mimics the stickiness of an object to skin. Tack testing is performed using a pull tester. A transdermal patch is placed on a test plate. The release liner is removed to expose the adhesive, and a metal cylinder probe is lowered until the probe touches the adhesive. The probe is slowly pulled up until the probe releases from the patch. The amount of effort required to break the adhesive bond between the patch and the probe is recorded.

Peel testing: Peel determines the force needed to break the bond between an adhesive and a surface at a fixed angle. Peel mimics the force required for removal of a transdermal patch. Peel testing is performed using a pull tester. A transdermal patch is applied to a test plate (adhesive side sticking to the test plate) and one end of the transdermal patch is clamped to a motorized pull gauge. The motor applies a force at a specific angle relative to the patch. The amount of effort required to separate the patch from the test plate is recorded.

Shear testing: Shear determines the ability of a material to resist lateral forces that cause the material to slide. Shear mimics the durability of an adhesive. Shear testing is performed using a pull tester. A transdermal patch is applied to a test plate (adhesive side sticking to the test plate) and one end of the transdermal patch is clamped to a motorized pull gauge. The motor applies a force laterally relative to the patch. The amount of effort required to separate the patch from the test plate is recorded.

Dissolution testing: Dissolution testing measures the rate of release of a therapeutic agent into a solution. A transdermal patch is secured to a support and submerged into a buffer solution. The buffer solution is stirred and maintained at a constant temperature. Aliquots of the buffer solution are sampled to measure the rate of release of the therapeutic agent from the transdermal patch.

A pharmaceutical composition described herein can comprise a transdermal patch, the transdermal patch comprising a SGLT2 inhibitor, wherein,
if in a study:
   a) the transdermal patch is placed into a first hydrochloric acid solution at an acidic pH;
   b) a profile at which the SGLT2 inhibitor migrates into the first hydrochloric acid solution over a period of 7 days is determined;
   c) an oral form of the SGLT2 inhibitor is placed into a second hydrochloric acid solution at an acidic pH; and
   d) a profile at which the SGLT2 inhibitor from the oral form migrates into the second hydrochloric acid solution over a period of 7 days is determined,
then the profile at which the SGLT2 inhibitor migrates into the first solution from the transdermal patch shows a total release at 7 days of less than a total release observed in the profile at which the SGLT2 inhibitor from the oral form migrates into the second solution within 12 hours.

In some embodiments, the basic pH is from about pH 7 to about pH 8. In some embodiments, the basic pH is about 7.1, about 7.2, about 7.3, about 7.4, about 7.5, about 7.6, about 7.7, about 7.8, about 7.9, or about 8.0. In some embodiments, the basic pH is about 7.2.

A pharmaceutical composition described herein can comprise a transdermal patch, the transdermal patch comprising an antidiabetic agent, wherein the transdermal patch exhibits a tack value of at least 5 grams, as determined by applying the transdermal patch to a stainless-steel cylinder, mechanically separating the transdermal patch from the stainless-steel cylinder, and measuring a level of effort required to separate the transdermal patch from the stainless-steel cylinder. In some embodiments, the transdermal patch exhibits a tack value of about 5 grams to about 100 grams. In some embodiments, the transdermal patch exhibits a tack value of about 5 grams to about 10 grams, about 5 grams to about 20 grams, about 5 grams to about 30 grams, about 5 grams to about 40 grams, about 5 grams to about 50 grams, about 5 grams to about 60 grams, about 5 grams to about 60 grams, about 5 grams to about 70 grams, about 5 grams to about 80 grams, or about 5 grams to about 90 grams. In some embodiments, the transdermal patch exhibits a tack value of about 5 grams, about 6 grams, about 7 grams, about 8 grams, about 9 grams, about 10 grams, about 11 grams about 12 grams, about 13 grams, about 14 grams, about 15 grams, about 16 grams, about 17 grams, about 18 grams, about 19 grams, about 20 grams, about 30 grams, about 40 grams, about 50 grams, about 60 grams, about 70 grams, about 80 grams, about 90 grams, or about 100 grams.

A pharmaceutical composition described herein can comprise a transdermal patch, the transdermal patch comprising an antidiabetic agent, wherein the patch exhibits a 90° peel adhesion of at least 50 grams, as determined by applying the transdermal patch to a steel plate, mechanically separating the transdermal patch from the steel plate by pulling the transdermal patch at a 90° angle, and measuring a level of effort required to separate the transdermal patch from the steel plate. In some embodiments, the transdermal patch exhibits a 90° peel adhesion of from about 50 grams to about 500 grams. In some embodiments, the transdermal patch exhibits a 90° peel adhesion of about 50 grams to about 100 grams, about 50 grams to about 150 grams, about 50 grams to about 200 grams, about 50 grams to about 250 grams, about 50 grams to about 300 grams, about 50 grams to about 350 grams, about 50 grams to about 400 grams, or about 50 grams to about 450 grams. In some embodiments, the transdermal patch exhibits a 90° peel adhesion of about 50 grams, about 100 grams, about 150 grams, about 200 grams, about 250 grams, about 300 grams, about 350 grams, about 400 grams, about 450 grams, or about 500 grams.

A pharmaceutical composition described herein can comprise a transdermal patch, the transdermal patch comprising an antidiabetic agent, wherein the patch exhibits a 180° peel adhesion of at least 50 grams, as determined by applying the transdermal patch to a steel plate, mechanically separating the transdermal patch from the steel plate by pulling the transdermal patch at a 180° angle, and measuring a level of effort required to separate the transdermal patch from the steel plate. In some embodiments, the transdermal patch exhibits a 180° peel adhesion of about 50 grams to about 100 grams, about 50 grams to about 150 grams, about 50 grams to about 200 grams, about 50 grams to about 250 grams, about 50 grams to about 300 grams, about 50 grams to about 350 grams, about 50 grams to about 400 grams, or about 50 grams to about 450 grams. In some embodiments, the transdermal patch exhibits a 180° peel adhesion of about 50 grams, about 100 grams, about 150 grams, about 200 grams, about 250 grams, about 300 grams, about 350 grams, about 400 grams, about 450 grams, or about 500 grams.

### Packaging of Transdermal Systems

The pharmaceutical compositions described herein can be packaged as a kit. In some embodiments, the present disclosure provides a kit comprising a transdermal system comprising a pharmaceutical composition disclosed herein and written instructions on use of the kit in the treatment of a disease.

In some embodiments, the present disclosure provides an article of manufacture comprising:
a. a transdermal patch comprising an antidiabetic agent;
b. aluminum packaging material; and
c. written instructions on use of the transdermal patch to treat diabetes.

In some embodiments, the written instructions can be, for example, a label. In some embodiments, the label can be approved by a regulatory agency. In some embodiments, the written instructions can suggest conditions or methods of administration. In some embodiments, the written instructions can provide a subject and a physician with guidance on achieving an optimal clinical outcome.

In some embodiments, the present disclosure provides a method comprising:
a. inserting a transdermal patch into an aluminum packaging material to provide a pouch,
wherein the transdermal patch comprises an antidiabetic agent; and
b. sealing the pouch.

In some embodiments, the aluminum packaging material protects the transdermal patch from heat. In some embodiments, the aluminum packaging material protects the transdermal patch from light.

In some embodiments, the sealing is of a perimeter of the pouch. In some embodiments, the sealing of the pouch is by heat. In some embodiments, the sealing of the pouch is at a temperature from about 100° C. to about 200° C. In some embodiments, the sealing of the pouch is at a temperature from about 100° C. to about 120° C., 100° C. to about 140° C., 100° C. to about 160° C., or about 100° C. to about 180° C. In some embodiments, the sealing of the pouch is at a temperature of about 100° C., about 120° C., about 140° C., about 160° C., about 180° C., or about 200° C.

Treatment of Subjects with Transdermal Systems

The pharmaceutical compositions described herein can be administered as soon as is practical after the onset of a disease or condition is detected or suspected, and for a length of time necessary for the treatment of the disease, such as, for example, from about 1 week to about 3 months. In some embodiments, the length of time a formulation can be administered can be about 1 day, about 2 days, about 3 days, about 4 days, about 5 days, about 6 days, about 1 week, about 2 weeks, about 3 weeks, about 4 weeks, about 1 month, about 2 months, about 3 months, about 6 months, about 1 year, about 2 years, about 3 years, about 5 years, or about 10 years. The length of treatment can vary for each subject.

Non-limiting examples of possible subjects for administration include the following. Subjects can be humans, non-human primates such as chimpanzees, and other apes and monkey species; farm animals such as cattle, horses, sheep, goats, and swine; domestic animals such as rabbits, dogs, and cats; and laboratory animals including rats, mice, and guinea pigs. A subject can be of any age. Subjects can be, for example, elderly adults, adults, adolescents, pre-adolescents, children, toddlers, and infants.

A subject described herein can have diabetes. A subject described herein can have Type 2 diabetes.

Pharmacokinetic parameters can be measured in a controlled study. In some embodiments, the following pharmacokinetic parameters can be used:
Cₘₐₓ—maximum observed concentration
Cₘᵢₙ—minimum observed concentration
Tₘₐₓ—time of occurrence of Cₘₐₓ
Tₘᵢₙ—time of occurrence of Cₘᵢₙ
Tlag—lag time, delay between drug administration and first observed drug concentration in plasma
AUC (0-t)—area under the concentration-time curve from pre-dose to defined time t
AUC(0-last)—area under the concentration-time curve from pre-dose to the last sample
AUC (0-inf)—area under the concentration-time curve extrapolated to infinite time
λz—terminal rate constant
t_{1/2}—terminal half-life
Cl/f—apparent oral clearance
Vz/F—apparent volume of distribution during terminal phase

In some embodiments, the controlled study comprises administering the pharmaceutical composition to a study subject, and after administering, collecting a blood sample from the study subject and measuring a plasma concentration of an antidiabetic agent in the blood sample.

In some embodiments, the blood samples are collected from the study subject before the administering. In some embodiments, the blood samples are collected from the study subject about 0.25, about 0.5, about 1, about 1.5, about 2, about 3, about 4, about 5, about 6, about 8, about 10, about 12, about 16, about 24, about 36, about 48, about 60, about 72, or about 96 hours after the administering. In some embodiments, the blood samples are collected from the study subject about 0.25, about 0.5, about 1, about 1.5, about 2, about 3, about 4, about 5, about 6, about 8, about 10, about 12, about 16, about 24, about 36, about 48, about 60, about 72, and about 96 hours after the administering. In some embodiments, the blood samples are collected from the study subject every day of the controlled study. In some embodiments, the blood samples are collected from the study subject before the administering, on the first day of the controlled study, on the mid-point of the controlled study, and on the last day of the controlled study. In some embodiments, the blood samples are collected from the study subject on the first day of the controlled study, on the mid-point of the controlled study, and on the last day of the controlled study.

In some embodiments, the controlled study further comprises administering a placebo to a control subject, and after the administering the placebo, collecting a control blood samples from the control subject, and measuring a plasma concentration of an antidiabetic agent in the control blood sample.

In some embodiments, the blood samples are collected from the study subject before the administering the placebo. In some embodiments, the control blood samples are collected from the study subject about 0.25, about 0.5, about 1, about 1.5, about 2, about 3, about 4, about 5, about 6, about 8, about 10, about 12, about 16, about 24, about 36, about 48, about 60, about 72, or about 96 hours after the administering the placebo. In some embodiments, the control blood samples are collected from the study subject about 0.25, about 0.5, about 1, about 1.5, about 2, about 3, about 4, about 5, about 6, about 8, about 10, about 12, about 16, about 24, about 36, about 48, about 60, about 72, and about 96 hours after the administering the placebo. In some embodiments, the blood samples are collected from the control subject every day of the controlled study. In some embodiments, the blood samples are collected from the control subject before the administering the placebo, on the first day of the controlled study, on the mid-point of the controlled study, and on the last day of the controlled study. In some embodiments, the blood samples are collected from the control subject on the first day of the controlled study, on the mid-point of the controlled study, and on the last day of the controlled study.

In some embodiments, a subject is administered a transdermal patch, wherein the transdermal patch comprises a SGLT2 inhibitor, wherein if, in a study, the transdermal patch is applied to a skin surface of a study subject, and the transdermal patch contains an initial amount of the SGLT2 inhibitor, and 7 days after the transdermal patch is applied to the skin surface of the study subject the transdermal patch is removed from the skin surface of the subject and is assayed for content of the SGLT2 inhibitor, then the content of the SGLT2 inhibitor is determined to be no greater than half the initial amount.

In some embodiments, a subject is administered a transdermal patch, wherein the transdermal patch comprises a SGLT2 inhibitor, wherein,
if in a study:
a) a first group of study subjects is administered the transdermal patch;
b) a mean Tmax of the SGLT2 inhibitor is determined for the first group of study subjects;
c) a second group of study subjects is administered an oral form of the SGLT2 inhibitor; and
d) a mean Tmax of the SGLT2 inhibitor is determined for the second group of study subjects,
then the mean Tmax of the SGLT2 inhibitor for the first group of study subjects is determined to be longer than the mean Tmax of the SGLT2 inhibitor for the second group of study subjects.

In some embodiments, a subject is administered a transdermal patch, wherein the transdermal patch comprises a SGLT2 inhibitor, wherein,
if in a study:
a) a first group of study subjects is administered the transdermal patch;
b) a mean Cmax of the SGLT2 inhibitor is determined for the first group of study subjects;
c) a second group of study subjects is administered an oral form of the SGLT2 inhibitor; and
d) a mean Cmax of the SGLT2 inhibitor is determined for the second group of study subjects,
then the mean Cmax of the SGLT2 inhibitor for the first group of study subjects is determined to be lower than the mean Cmax of the SGLT2 inhibitor for the second group of study subjects.

The present invention includes a transdermal patch containing: at least one SGLT2 inhibitor or antidiabetic agent; an adhesive layer containing an acrylic adhesive in which the SGLT2 inhibitor and/or or antidiabetic agent is incorporated; optionally, one or more permeation enhancers in the adhesive layer, to facilitate delivery of the SGLT2 inhibitor and/or or antidiabetic agent through the skin; a removeable release liner, positioned on one surface of the adhesive layer, configured to be removed prior to application; and a backing film on the opposed surface of the adhesive layer, impermeable or occlusive to protect the patch and control drug release direction; wherein the adhesive layer is positioned between the removable release liner and the backing film, such that upon removal of the liner and placement on a skin surface, drug permeation is directed outward from the adhesive-contacting layer toward the skin.

In specific embodiments, the SGLT2 inhibitor includes canagliflozin, dapagliflozin, empagliflozin, ertugliflozin, ipragliflozin, luseogliflozin, tofogliflozin, a solvate or hydrate thereof.

In specific embodiments, the SGLT2 inhibitor includes canagliflozin, dapagliflozin, empagliflozin, ertugliflozin, ipragliflozin, luseogliflozin, tofogliflozin, a solvate or hydrate thereof.

In specific embodiments, the antidiabetic agent inhibitor includes alpha-glucosidase inhibitors (acarbose, miglitol); amylin analogue (pramlintide); dipeptidyl peptidase 4 (DPP-4) inhibitor (alogliptan, linagliptin, saxagliptin, sitagliptin); incretin mimetics/GLP-1 agonist (albiglutide, dulaglutide, exenatide, liraglutide, lixisenatide); meglitinide (nateglinide, repaglinide), metformin (biguanide), sulfonylureas (chlorpropamide, glimepiride, glipizide, glyburide, tolazamide, tolbutamide), thiazolidinedione (rosiglitazone, pioglitazone), and insulin.

In specific embodiments, the adhesive layer contains an acrylic adhesive functionalized with the crosslinking groups carboxyl (—COOH) and/or hydroxyl (—OH)).

In specific embodiments, the permeation enhancer includes organic acids, nonionic surfactants, or fatty acid derivatives.

In specific embodiments, the permeation enhancer includes the organic acid levulinic acid or lactic acid; the nonionic surfactant polysorbate 80; or fatty acid derivatives.

In specific embodiments, the removeable release liner includes a polyester film or fluoropolymer-coated polyester.

In specific embodiments, the backing film includes polyethylene monolayer film, polyester, or laminate of polyethylene and aluminum vapor-coated polyester.

In specific embodiments, the SGLT2 inhibitor is physically and/or chemically integrated into the adhesive matrix, uniformly dispersed or dissolved within the acrylic adhesive.

In specific embodiments, the SGLT2 inhibitor is further mixed solvent.

In specific embodiments, the SGLT2 inhibitor is further mixed the solvent methanol, ethyl acetate, or combination thereof.

In specific embodiments, the adhesive layer further includes one or more additional excipients, stabilizers, carriers, diluents, dispersants, thickeners, or agents for manufacturability or stability.

In specific embodiments, the patch has a surface area of from about 1 square centimeter to 100 square centimeters.

In specific embodiments, the patch has a thickness from about 0.1 millimeter to about 10 millimeters.

In specific embodiments, the patch has a mass of from about 10 milligrams to about 10 grams.

In specific embodiments, the adhesive layer includes a single, monolithic drug-in-adhesive layer and contains no more than one drug-in-adhesive layer, thereby increasing manufacturing reproducibility and patch consistency.

In specific embodiments, the patch is configured to provide sustained release of the SGLT2 inhibitor or antidiabetic agent over a period of at least 7 days upon application to the skin.

In specific embodiments, the application to human skin for up to 7 days delivers at least 50% by weight of the total SGLT2 inhibitor or antidiabetic agent content measured by residual analysis post-removal.

In specific embodiments, the patch demonstrates effective transdermal delivery of SGLT2 inhibitors, as confirmed by skin permeation testing using human or animal skin, yielding unexpectedly efficient delivery despite the moderate polarity and molecular weight of the SGLT2 inhibitor.

In specific embodiments, the SGLT2 inhibitor is released from the patch in a controlled, sustained manner over 7 days in an acidic environment, wherein the total release after 7 days is less than the total release from an orally administered form after 12 hours, resulting in reduction of peak-related systemic side effects.

In specific embodiments, the patch is configured to exhibit a tack value of at least 5 grams, measured by force required to separate the patch from a stainless-steel cylinder.

In specific embodiments, the patch exhibits a 90° peel adhesion of at least 50 grams, as measured by force required to pull the patch from a steel plate at a 90° angle.

In specific embodiments, the patch exhibits a 180° peel adhesion of at least 50 grams, as measured by force required to pull the patch from a steel plate at a 180° angle.

In specific embodiments, the patch does not exhibit significant cold flow or edge tack under ambient storage, as determined by visual inspection after die-cutting and storage.

In specific embodiments, the patch is free of visible crystal formation or phase separation during extended storage or after freeze-thaw cycling, when formulated without glycerol, glycol, sodium dodecyl sulfate, or Tween 20.

In specific embodiments, the patch maintains greater than 95% purity of the SGLT2 inhibitor after at least 45 days storage at 37° C., as measured by HPLC, with no major impurities detected.

In specific embodiments, the release liner and backing films are each made from commercially available medical-grade films, enabling regulatory approval and scalable manufacturing.

In specific embodiments, the patch is packaged in a sealed aluminum pouch, the pouch being heat-sealed and configured to protect the patch from light and moisture during storage and shipping.

In specific embodiments, the patch is designed to exhibit robust adhesion to skin, minimal lifting, and low irritation or redness after 24 hours of continuous wear, as demonstrated in human wear studies.

In specific embodiments, the residual solvent levels in the patch are less than 500 ppm for methanol, ethanol, or ethyl acetate upon release for commercial distribution.

In specific embodiments, the SGLT2 inhibitor is present in an amount from about 5% to about 50% by weight of the total solids content of the transdermal patch.

In specific embodiments, the acrylic adhesive is an acrylate polymer including one or more monomers selected from acrylate, methacrylate, methyl methacrylate, acrylic acid, or vinyl acetate.

In specific embodiments, the adhesive is functionalized with a crosslinking agent selected from carboxylic acid or alcohol functional groups.

In specific embodiments, the permeation enhancer is selected from the group consisting of organic acids, fatty acid esters, nonionic surfactants, and terpenes.

In specific embodiments, the permeation enhancer is levulinic acid, lactic acid, or polysorbate 80.

In specific embodiments, the adhesive layer further includes a solvent selected from methanol, ethyl acetate, dimethylsulfoxide, or heptane.

In specific embodiments, the surface area is between about 5 cm² and about 30 cm², and the thickness is between about 0.1 mm and about 0.25 mm.

In specific embodiments, the patch includes a release liner made of a fluoropolymer-coated polyester film.

In specific embodiments, the backing film is a polyethylene monolayer or an aluminum vapor-coated polyester film.

In specific embodiments, the patch exhibits a tack time of at least about 5 minutes under a pressure of at least about 7 grams as determined by a pull tester.

In specific embodiments, the patch demonstrates no visible crystallization or cold flow under freeze-thaw cycles or prolonged storage at 37° C.

In specific embodiments, the SGLT2 inhibitor is dapagliflozin propane diol hydrate and the patch provides sustained release for at least 7 days in skin permeation studies.

In specific embodiments, the patch delivers at least 50% of the total SGLT2 inhibitor content to the subject during the intended wear period as measured by post-use residual analysis.

In specific embodiments, after use and removal of the patch from skin after 24 hours, no significant redness, irritation, or lifting is observed in at least 90% of human subjects tested.

In specific embodiments, the patch is packaged in an aluminum pouch that is heat-sealed at a temperature of 100° C. to 200° C.

In specific embodiments, the patch includes a single, uniform drug-in-adhesive matrix layer.

In specific embodiments, the written instructions recommend application to the arm, abdomen, or thigh and replacement weekly.

In specific embodiments, the patch when applied provides a Tmax of the SGLT2 inhibitor greater than 5 hours and a Cmax lower than that observed with an equivalent oral dose.

In specific embodiments, the transdermal patch maintains at least 95% drug purity after 45 days of storage at 37° C.

The present invention also includes a kit containing: a transdermal patch described herein; an aluminum packaging material enclosing the transdermal patch; and written instructions for use of the transdermal patch to treat diabetes.

In specific embodiments, the kit further includes aluminum packaging material configured to provide a sealed pouch enclosing the transdermal patch.

In specific embodiments, the kit further includes aluminum packaging material configured to provide a sealed pouch enclosing the transdermal patch and wherein the aluminum packaging material is sealed at the perimeter to protect the transdermal patch from moisture and oxygen ingress.

In specific embodiments, the kit further includes aluminum packaging material configured to provide a sealed pouch enclosing the transdermal patch and wherein the aluminum packaging material is configured to be heat sealed at temperatures between about 100° C. and about 200° C.

In specific embodiments, the kit further includes aluminum packaging material configured to provide a sealed pouch enclosing the transdermal patch and wherein the aluminum pouch is light-impermeable, thereby protecting the patch from light-induced degradation.

In specific embodiments, the kit further includes written instructions for use of the transdermal patch to treat diabetes.

In specific embodiments, the kit further includes written instructions for use of the transdermal patch to treat diabetes and wherein the written instructions are in the form of a regulatory-approved label specifying indications, dosage, and administration.

In specific embodiments, the kit further includes written instructions for use of the transdermal patch to treat diabetes and wherein the written instructions include guidance on patch application sites and schedule for re-application.

In specific embodiments, the kit further includes a batch or lot number label affixed to the packaging.

In specific embodiments, the instructions include information on storage conditions for product stability, including recommended temperature and humidity.

In specific embodiments, the kit further includes a carton or outer box containing the aluminum pouch and the written instructions.

In specific embodiments, the packaging material is that includes of multi-layer laminate including aluminum foil and thermoplastic resin for improved barrier properties.

In specific embodiments, the instructions include contraindications, potential adverse effects, and emergency contact information.

In specific embodiments, the kit further includes a user scoring tool or template to assist in safe removal of the patch from the pouch.

In specific embodiments, the aluminum pouch is embossed with product information including product name and lot number.

In specific embodiments, the carton is printed with directions for disposal of used patches and packaging in accordance with regulatory requirements.

The present invention also includes a method of treating diabetes in a subject in need thereof, the method including: administering to the subject a transdermal patch, the transdermal patch including a mixture of a SGLT2 inhibitor and an acrylic adhesive, wherein the transdermal patch is applied to a skin surface of the subject in an amount effective to treat diabetes.

In specific embodiments, the subject is a human.

In specific embodiments, the subject is an adult human.

In specific embodiments, the subject is a pediatric human.

In specific embodiments, the subject is elderly.

In specific embodiments, the method treats Type 2 diabetes.

In specific embodiments, the method treats hyperglycemia.

In specific embodiments, the method reduces fasting blood glucose in the subject.

In specific embodiments, the method reduces postprandial blood glucose in the subject.

In specific embodiments, the method further includes monitoring the blood glucose level of the subject during patch application.

In specific embodiments, the method further includes adjusting the frequency of patch replacement based on the subject's glycemic response.

In specific embodiments, the method reduces the occurrence of hypoglycemic events compared to oral administration of the antidiabetic agent.

In specific embodiments, the method mitigates gastrointestinal adverse effects associated with oral SGLT2 inhibitor administration.

In specific embodiments, the method reduces hepatic first-pass metabolism of the antidiabetic agent.

In specific embodiments, the method mitigates the risk of urinary tract infections associated with SGLT2 inhibitor administration.

In specific embodiments, the method further includes advising the subject to rotate the site of patch application.

In specific embodiments, the method further includes removing the patch after 7 days and applying a new patch to a different area of skin.

In specific embodiments, the method further includes counseling the subject regarding signs of skin irritation.

In specific embodiments, the method includes the periodic evaluation of patch adhesion and subject skin at the application site.

In specific embodiments, the method treats insulin resistance in the subject.

In specific embodiments, the method lowers the subject's glycated hemoglobin (HbA1c) over a period of at least 3 months.

In specific embodiments, the patch is administered to a subject and replaced at least once every 7 days.

In specific embodiments, the antidiabetic agent inhibitor includes alpha-glucosidase inhibitors (acarbose, miglitol); amylin analogue (pramlintide); dipeptidyl peptidase 4 (DPP-4) inhibitor (alogliptan, linagliptin, saxagliptin, sitagliptin); incretin mimetics/GLP-1 agonist (albiglutide, dulaglutide, exenatide, liraglutide, lixisenatide); meglitinide (nateglinide, repaglinide), metformin (biguanide), sulfonylureas (chlorpropamide, glimepiride, glipizide, glyburide, tolazamide, tolbutamide), thiazolidinedione (rosiglitazone, pioglitazone), and insulin.

In certain embodiments, the present invention provides transdermal patches including SGLT2 inhibitors (such as dapagliflozin) as an alternative to traditional oral administration, conferring the advantages of minimally invasive delivery and bypass of first-pass hepatic metabolism.

In some embodiments, the present invention provides transdermal patches having a single drug-in-adhesive layer, wherein the drug-in-adhesive layer includes a mixture of an SGLT2 inhibitor and an adhesive. This single-layer design facilitates improved manufacturing reproducibility, enhanced stability, and optimized transdermal performance compared to multi-layer systems.

In certain embodiments, the transdermal patches described herein are configured to provide sustained release of an SGLT2 inhibitor, such as dapagliflozin, over a period of at least 7 days, as confirmed by human cadaver skin permeation studies, thus enabling weekly dosing in contrast to daily oral tablet regimens.

In further embodiments, acidic pH dissolution studies demonstrate that the release profile of the SGLT2 inhibitor from the patch is slower and more controlled over 7 days compared to the rapid and near-complete release from oral forms within 12 hours. This sustained, controlled release is anticipated to reduce peak-related side effects and allow less frequent dosing.

In some embodiments, skin permeation testing confirms effective delivery of dapagliflozin across human skin from the patch matrix, establishing feasibility for transdermal delivery—a result seen as surprising for this drug class, given the moderate molecular weight and polarity of many antidiabetic agents.

In certain embodiments, the patches are designed to deliver at least 50 wt. % of the SGLT2 inhibitor content during the wear period (as assessed by residual drug analysis post-wear), supporting clinical utility and commercial feasibility.

In some embodiments, the present invention encompasses patches including acrylic adhesives, optionally functionalized with crosslinking groups such as —COOH or —OH, which are compatible with SGLT2 inhibitors and demonstrate desirable tack, peel, and shear characteristics for skin adherence and easy removal without irritation.

In certain embodiments, the transdermal patches incorporate permeation enhancers such as organic acids (e.g., levulinic acid) and/or nonionic surfactants (e.g., polysorbate 80) to improve transdermal drug flux and provide extended, stable release kinetics.

In some embodiments, the present invention provides transdermal patches with specified parameters, including a surface area of about 1-100 cm², thickness of about 0.1-10 mm, mass of about 10 mg-10 g, and defined adhesive performance specifications (tack, peel, shear), offering broad flexibility for commercial application and scalable manufacturing.

In specific embodiments, the present invention provides for transdermal patches that do not exhibit significant cold flow or crystallization when formulated without excipients like glycerol, glycol, sodium dodecyl sulfate (SDS), or Tween 20—phenomena verified by freeze-thaw and storage studies—which can be significant for product integrity during storage and shipping.

In certain embodiments, pharmacokinetic analyses show that TDS patches yield a longer Tmax and lower Cmax in vivo as compared to oral formulations, supporting the benefit of reduced plasma peaks/troughs, steadier systemic exposure, and potentially fewer hypoglycemic events.

In some embodiments, the present invention provides patches utilizing commercially available release liners and backing films (e.g., 3M Scotchpak™ 9744, 9720, and others), which supports regulatory approval, batch consistency, and seamless scale-up for manufacturing.

In further embodiments, the present invention enables cost-effective, scalable manufacturing and packaging, including process steps such as solution-coating, drying, lamination, die-cutting, and packaging in sealed aluminum pouches to maintain product stability during distribution and storage.

In certain embodiments, the present invention demonstrates that the patches provide robust adhesion, minimal lifting, and low irritation or redness upon wear, as shown in human and animal use studies, even under challenging use conditions; such attributes are paramount for patient adherence and commercial acceptance.

In additional embodiments, present stability studies indicate that the transdermal patches maintain greater than 95% dapagliflozin purity after prolonged storage, with no major impurities detected, aligning with regulatory expectations for pharmaceutical quality and ensuring consistent efficacy and safety.

In specific embodiments, the present invention provides a pharmaceutical composition including a transdermal patch, the transdermal patch including a mixture of SGLT2 inhibitor and an acrylic adhesive.

In specific embodiments, the present invention provides a pharmaceutical composition including a transdermal patch, the transdermal patch including one and no more than one drug-in-adhesive layer, wherein the drug-in-adhesive layer includes a mixture of a SGLT2 inhibitor and an adhesive.

In specific embodiments, the present invention provides a pharmaceutical composition including a transdermal patch, the transdermal patch including a SGLT2 inhibitor, wherein the transdermal patch has a surface area of from about 1 square centimeter to about 100 square centimeters.

In specific embodiments, the present invention provides a pharmaceutical composition including a transdermal patch, the transdermal patch including a SGLT2 inhibitor, wherein the transdermal patch has a thickness from about 0.1 millimeter to about 10 millimeters.

In specific embodiments, the present invention provides a pharmaceutical composition including a transdermal patch, the transdermal patch including an antidiabetic agent, wherein the transdermal patch has a mass of from about 10 milligrams to about 10 grams.

In specific embodiments, the transdermal patch exhibits a tack value of at least 5 grams, as determined by applying the transdermal patch to a stainless-steel cylinder, mechanically separating the transdermal patch from the stainless-steel cylinder, and measuring a level of effort required to separate the transdermal patch from the stainless-steel cylinder.

In specific embodiments, the transdermal patch exhibits a 90° peel adhesion of at least 50 grams, as determined by applying the transdermal patch to a steel plate, mechanically separating the transdermal patch from the steel plate by pulling the transdermal patch at a 90° angle, and measuring a level of effort required to separate the transdermal patch from the steel plate.

In specific embodiments, the transdermal patch exhibits a 180° peel adhesion of at least 50 grams, as determined by applying the transdermal patch to a steel plate, mechanically separating the transdermal patch from the steel plate by pulling the transdermal patch at a 180° angle, and measuring a level of effort required to separate the transdermal patch from the steel plate.

In specific embodiments, if in a study the transdermal patch is applied to cadaver skin and permeation of the SGLT2 inhibitor across the cadaver skin is monitored, then a permeation profile of at least 7 days is observed.

In specific embodiments, if in a study: (a) the transdermal patch is placed into a first hydrochloric acid solution at an acidic pH; (b) a profile at which the SGLT2 inhibitor migrates into the first hydrochloric acid solution over a period of 7 days is determined; (c) an oral form of the SGLT2 inhibitor is placed into a second hydrochloric acid solution at the acidic pH; and (d) a profile at which the SGLT2 inhibitor from the oral form migrates into the second solution over a period of 7 days is determined, then the profile at which the SGLT2 inhibitor migrates into the first solution from the transdermal patch shows a total release at 7 days of less than a total release observed in the oral form within 12 hours.

In specific embodiments, if in a study: (a) the transdermal patch is placed into a sodium phosphate solution at a basic pH; and (b) a profile at which the SGLT2 inhibitor migrates into the solution is determined, then the profile at which the SGLT2 inhibitor migrates into the solution from the transdermal patch shows a total release of SGLT2 inhibitor over a period of at least about 7 days.

In specific embodiments, the present invention provides a pharmaceutical composition including a mixture of a SGLT2 inhibitor and an acrylic adhesive.

In specific embodiments, the present invention provides a method of treating a condition, the method including administering to a subject in need thereof a transdermal patch, the transdermal patch including a SGLT2 inhibitor, wherein the transdermal patch delivers the SGLT2 inhibitor over a period of at least about 7 days.

In specific embodiments, the present invention provides a method of treating a condition, the method including administering to a subject in need thereof a therapeutically-effective amount of an antidiabetic agent via a transdermal patch, the transdermal patch including the antidiabetic agent, wherein the transdermal patch has a mass from about 10 milligrams to about 10 grams.

In specific embodiments, the present invention provides a method of treating Type 2 diabetes, the method including applying a transdermal patch to a subject in need thereof, wherein the transdermal patch includes a therapeutically-effective amount of an antidiabetic agent, and is applied to a skin surface of a subject.

In specific embodiments, the present invention provides a method including: (a) contacting a skin surface of a subject in need thereof with a first sample of a mixture, the mixture including a therapeutically-effective amount of an antidiabetic agent and an adhesive; and (b) 7 days after the contacting, contacting the subject with a second sample of the mixture.

In specific embodiments, the present invention provides a method of treating Type 2 diabetes, the method including contacting a skin surface of a subject in need thereof with a mixture, the mixture including a therapeutically-effective amount of an antidiabetic agent and an adhesive, wherein the antidiabetic agent has a molecular weight of less than about 1000 Daltons.

In specific embodiments, if in a study the transdermal patch is applied to a study subject, then the study subject exhibits a Tmax of the antidiabetic agent at least 5 hours after the transdermal patch is applied to the study subject.

In specific embodiments, the transdermal patch delivers to the subject at least 50% of the SGLT2 inhibitor in the transdermal patch.

In specific embodiments, the present invention provides a method of preparing a formulation, the method including: (a) contacting a solvent with a SGLT2 inhibitor to provide a first mixture; (b) contacting the first mixture with a permeation enhancer to provide a second mixture; and (c) contacting the second mixture with an acrylic adhesive to provide the formulation.

In specific embodiments, the present invention provides a method of preparing a formulation, the method including: (a) contacting a solvent with a SGLT2 inhibitor to provide a first mixture; and (b) contacting the first mixture with an acrylic adhesive to provide the formulation.

In specific embodiments, the present invention provides a method of preparing a transdermal patch, the method including: (a) combining a SGLT2 inhibitor, an adhesive, and a solvent to provide a crude mixture; and (b) preparing a uniform mixture by stirring the crude mixture until it becomes substantially uniform.

In specific embodiments, the present invention provides a method including applying a mixture to a release liner, wherein the mixture includes a SGLT2 inhibitor and an adhesive, and the release liner is a polyester film.

In specific embodiments, the present invention provides a method including applying a transdermal patch to a subject, the transdermal patch including: (a) a release liner; and (b) a mixture of SGLT2 inhibitor and an adhesive.

In specific embodiments, the present invention provides a method including inserting a transdermal patch into an aluminum packaging material to provide a pouch, wherein the transdermal patch includes an antidiabetic agent; and sealing the pouch.

In specific embodiments, the present invention provides an article of manufacture including: (a) a transdermal patch including an antidiabetic agent; (b) aluminum packaging material; and (c) written instructions on use of the transdermal patch to treat diabetes.

### EXAMPLES

Non-limiting examples of compounds, adhesives, enhancers, release liners, and backing films disclosed herein are listed in TABLE 1.

TABLE 1

| Name | Structure or Description |
|---|---|
| Dapagliflozin (shown here in the (S)- propane-1,2- diol (1:1) monohydrate form) | 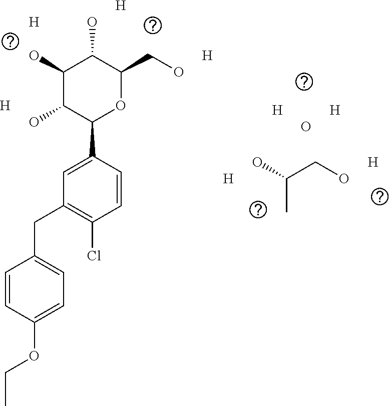 |
| | (1S)-1,5-Anhydro-1-C-[4-chloro-3-[(4- |
| | ethoxyphenyl)methyl]phenyl]-D-glucitol (S)-propane-1,2-diol (1:1) |
| | monohydrate |
| | C₂₁H₂₅ClO₆•C₃H₈O₂•H₂O |
| GELVA ® | acrylates copolymer, contains vinyl acetate, contains —OH functional |
| GMS 788 | groups, 41% solids basis, 5250 cP viscosity |
| DURO-TAK ® | acrylates copolymer, contains vinyl acetate, contains —COOH |
| 87-2051 | functional groups, 51.5% solids basis, 4000 cP viscosity |
| DURO-TAK ® | acrylates copolymer, contains vinyl acetate, contains —COOH |
| 87-2052 | functional groups, contains crosslinker, 47.5% solids basis, 2750 cP |
| | viscosity |
| DURO-TAK ® | acrylates copolymer, contains —COOH/—OH functional groups, |
| 87-2074 | contains crosslinker, 29.5% solids basis, 1500 cP viscosity |
| DURO-TAK ® | acrylates copolymer, contains vinyl acetate, contains —OH functional |
| 87-2516 | groups, contains crosslinker, 41.5% solids basis, 4350 cP viscosity |
| DURO-TAK ® | acrylates copolymer, contains —COOH functional groups, contains |
| 87-2852 | crosslinker, 44.5% solids basis, 2500 cP viscosity |
| DURO-TAK ® | acrylates copolymer, contains vinyl acetate, contains —COOH |
| 87-4287 | functional groups, 39% solids basis, 8000 cP viscosity |
| Silbione ® RT | silicone adhesive, two component silicone elastomers that crosslink |
| GEL 4717A, | at room temperature |
| 4717B | |
| OPPANOL ® B12 | polyisobutene, 56% solids basis |
| DURO-TAK ® | polyisobutene, 38% solids basis, 6000 cP viscosity |
| 87-6908 | |
| Liveo ™ BIO- | silicone adhesive, supplied in ethyl acetate, 60% solids basis, |
| PSA 7-4502 | medium tack |
| Liveo ™ BIO- | silicone adhesive, supplied in ethyl acetate, 60% solids basis, high |
| PSA 7-4602 | tack |
| Liveo ™ BIO- | silicone adhesive, supplied in heptane, 70% solids basis, high tack |
| PSA 7-4301 | |
| glycerol | 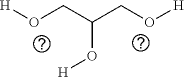 |
| | propane-1,2,3-triol |
| | C₃H₈O₃ |
| propylene glycol | 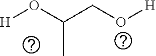 |
| | propane-1,2-diol |
| | C₃H₈O₂ |
| polyethylene | polyether, H—(O—CH₂—CH₂)ₙ—OH |
| glycol | |
| diethylene glycol monoethyl ether Transcutol ® | 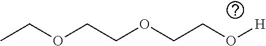 |
| | 2-(2-ethoxyethoxy)ethanol |
| | C₆H₁₄O₃ |
| SDS | 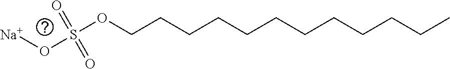 |
| | sodium dodecyl sulfate |
| | NaSO₄C₁₂H₂₅ |
| D-limonene | 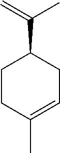 |
| | (4R)-1-methyl-4-prop-1-en-2-ylcyclohexene |
| | C₁₀H₁₆ |
| polysorbate 20 Tween ® 20 | 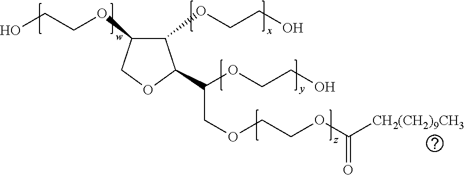 |
| polysorbate 80 | 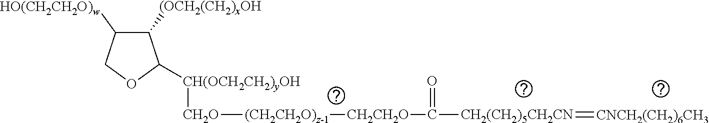 |
| castor oil | 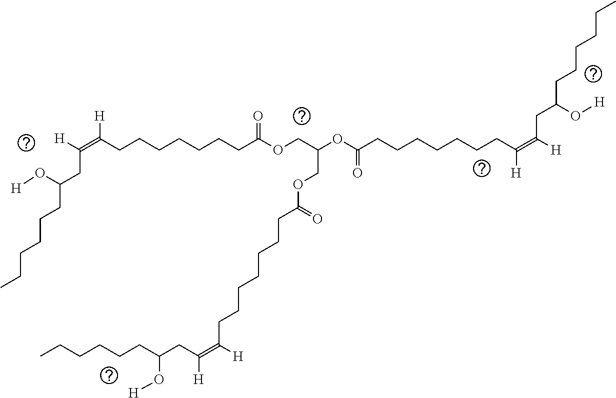 [SMILES: CCCCCCC(O)C/C=C\CCCCCCCC(=O)OCC(COC(=O)CCCCCCC/C=C\CC(O)CCCCCC)OC(=O)CCCCCCC/C=C\CC(O)CCCCCC] |
| | 2,3-bis[[(Z)-12-hydroxyoctadec-9-enoyl]oxy]propyl (Z)-12- |
| | hydroxyoctadec-9-enoate |
| | C₅₇H₁₀₄O₉ |
| Kollidon ® VA 64 | copovidone, copolymer of vinylpyrrolidone and vinyl acetate, |
| | soluble in water and alcohols |
| Plasdone ™ S-630 | copovidone, 60:40 linear, random copolymer of vinylpyrrolidone and |
| | vinyl acetate |
| Plasdone ™ C-30 | plasdone povidone, water-soluble linear homopolymer of |
| | vinylpyrrolidone |
| levulinic acid | 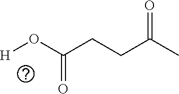 |
| | 4-oxopentanoic acid |
| | C₅H₈O₃ |
| lactic acid | 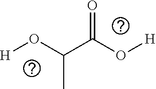 |
| | 2-hydroxypropanoic acid |
| | C₃H₆O₃ |
| Crodamol ™ | 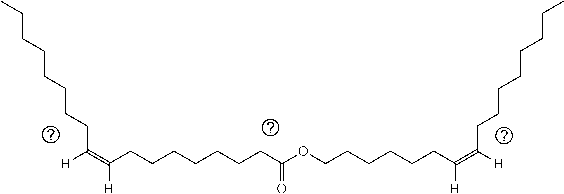 [SMILES: CCCCCCCC/C=C\CCCCCCCC(=O)OCCCCCC/C=C\CCCCCCCC] |
| | oleyl oleate |
| | C₃₆H₆₈O₂ |
| eucalyptus oil | contains 1,8-cineol |
| Polyplasdone ™ | crospovidone, crosslinked homopolymer of N-vinyl-2-pyrrolidone, |
| XL | 110-140 micron average particle size |
| Prosolv ® 730 | composite made from microcrystalline cellulose, silicon dioxide, and |
| | copovidone |
| Plastoid ® B | copolymer of butylmethacrylate and methylmethacrylate |
| Kollidon ® CL | crosslinked polyvinylpyrrolidone |
| polyvinyl- pyrrolidone | 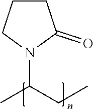 |
| Povidone K90 | polyvinylpyrrolidone, K value of about 81.0 to about 97.2 |
| Povidone K-17 | polyvinylpyrrolidone, K value of about 15.3 to about 18.4 |
| Povidone K29/32 | polyvinylpyrrolidone, K value of about 29 to about 32 |
| Loparex 4400 | polyester film, made from biaxially stretched polyethylene |
| | terephthalate, high tensile strength, high heat resistance |
| 3M Scotchpak ™ | fluoropolymer coated polyester film, transparent, occlusive, ≤100 |
| 9744 | g/in liner release, 70 lbs/in cross direction tensile strength, 60 lbs/in |
| | machine direction tensile strength |
| 3M Scotchpak ™ | laminate of tan pigmented polyethylene, thermoplastic resin, and |
| 9738 | aluminum vapor coated polyester |
| 3M CoTran ™ | polyethylene monolayer film, translucent, 3 lbs/in cross direction |
| 9720 | tensile strength, 5 lbs/in machine-direction tensile strength, corona |
| | treated on both sides |
| 3M CoTran ™ | polyurethane backing film |
| 9701 | |
| 3M Scotchpak ™ | laminate of tan pigmented polyethylene and aluminum vapor coated |
| 1109 | polyester |
| 3M Scotchpak ™ | fluoropolymer coated polyester film, transparent, occlusive, ≤100 |
| 1022 | g/in liner release, 110 lbs/in cross direction tensile strength, 90 lbs/in |
| | machine direction tensile strength |

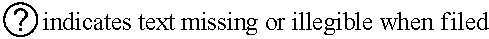

### Example 1: Solubility of Dapagliflozin in Adhesive-Compatible Solvents

Dapagliflozin was added to various solvents to determine solubility and compatibility with polyisobutene (PIB) or acrylic adhesive systems. Dapagliflozin was added to a test tube followed by the selected solvent, with visual observations noted upon addition of solvent and 24 hours later. Experimental details are summarized in TABLE 2.

TABLE 2

| | Solvent | Dapagliflozin | Observations - | Observations - |
|---|---|---|---|---|
| Solvent | (mL) | (mg) | Initial | 24 hours |
| Ethyl | 2 | 22.8 | soluble, clear | in solution, |
| Acetate | | | | no color change |
| MeOH | 2 | 88.9 | soluble, clear | in solution, |
| | | | | no color change |
| DMSO | 2 | 22 | soluble, clear | in solution, |
| | | | | no color change |
| Heptane | 2 | 25.1 | soluble, clear | not in solution, |
| | | | | clumpy |
| D.I. | 2 | 24.9 | insoluble | not in solution, |
| H₂O | | | | no color change |

The solubility studies in TABLE 2 determined that both PIB adhesives (which commonly use heptane or less frequently methanol) and acrylic adhesives (which commonly use ethyl acetate or less frequently methanol) can be used with dapagliflozin. Based on these results, a suspension is the basis for a formulation with PIB adhesives and a solution is the basis for a formulation with acrylic adhesives.

### Example 2: Compositions of Dapagliflozin Formulations

Formulations containing different types of adhesives and optional enhancers were developed for the drug in adhesive layer of the TDS patch. TABLES 3-5 describe the compositions of formulations containing mixtures of dapagliflozin, solvent, and adhesive. TABLES 4-14 describe the compositions of formulations containing mixtures of dapagliflozin, solvent, adhesive, and enhancer.

TABLE 3

| | 3A | | 3B | | 3C | |
|---|---|---|---|---|---|---|
| | Calc. Mass | Obsv. Mass | Calc. Mass | Obsv. Mass | Calc. Mass | Obsv. Mass |
| Ingredients | (grams) | (grams) | (grams) | (grams) | (grams) | (grams) |
| B12 (12A) | 4 | 4.0038 | | | | |
| Heptane | 1 | 1.0414 | | | | |
| Dapagliflozin | 0.4 | 0.4051 | 0.4 | 0.4157 | 0.4 | 0.40015 |
| 2516 | | | 4 | 4.1634 | | |
| Methanol | | | 1 | 1.069 | | |
| 2052 | | | | | 4 | 4.0034 |
| Isopropyl | | | | | 1 | 1.002 |
| Alcohol | | | | | | |
| 2074 | | | | | | |
| Total (g) | 5.4 | 5.4503 | 5.4 | 5.6481 | 5.4 | 5.40555 |
| Total Solids | 2.6 | | 2.1 | | 1.6 | |
| (g) | | | | | | |
| % Solids | 48.89 | | 38.15 | | 29.26 | |
| % Drug | 15.2 | | 19.4 | | 25.3 | |

Manufacturing parameters for patches from formulations 3A-C:
Release liner (3A): 4400
Release liner (3B. 3C): 9744
Backing film: 1109
Thickness (drug in adhesive coated to the release liner): 0.013 inches
Coating speed: 9 rpm
Drying temperatures: 80° C. (first stage) and 120° C. (second stage)

TABLE 4

| | 7A | | 7B | | 7C | | 7D | |
|---|---|---|---|---|---|---|---|---|
| | Calc. | Obsv. | Calc. | Obsv. | Calc. | Obsv. | Calc. | Obsv. |
| | Mass | Mass | Mass | Mass | Mass | Mass | Mass | Mass |
| Ingredients | (grams) | (grams) | (grams) | (grams) | (grams) | (grams) | (grams) | (grams) |
| 2051 | 4 | 4.07 | 4 | 4.14 | | | | |
| 6908 | | | | | 4 | 5.5 | | |
| 4287 | | | | | | | 4 | 4.45 |
| Methanol | | | | | | | 1 | 1.15 |
| Ethyl Acetate | | | 1 | 1.82 | | | | |
| Heptane | | | | | 1 | 1.58 | | |
| Dapagliflozin | 0.5 | 0.51 | 0.5 | 0.51 | 0.5 | 0.51 | 0.5 | 0.48 |
| Total (g) | 5.5 | 4.580 | 5.5 | 6.470 | 5.5 | 7.590 | 5.5 | 6.090 |
| % Drug | | 19.6 | | 19.3 | | 19.6 | | 22.0 |

Manufacturing parameters for patches from formulations 7A-D:
Release liner: 4400
Backing film: 1109
Thickness (drug in adhesive layer coated to the release liner): 0.015 inches
Coating speed: 7 rpm
Drying temperatures: 80° C. (first stage) and 120° C. (second stage)

TABLE 5

| | 23A | 23B | 23C |
|---|---|---|---|
| Ingredients | Obsv. Mass (grams) | | |
| 4502 | 2.03 | | |
| 4602 | | 2.01 | |
| 4301 | | | 2.04 |
| Ethyl Acetate | 1.12 | 1.1 | |
| Heptane | | | 1.03 |
| Dapagliflozin | 0.1982 | 0.2058 | 0.1967 |
| Total (g) | 3.3482 | 3.3158 | 3.2667 |

Manufacturing parameters for patches from formulations 23A-C:
Release liner: 9744
Backing film: 9738
Thickness (drug in adhesive layer coated to the release liner): 0.015 inches
Coating speed: 8 rpm
Drying temperatures: 80° C. (first stage) and 120° C. (second stage)

TABLE 6

| | 8A | | 8B | | 8C | | 8D | |
|---|---|---|---|---|---|---|---|---|
| | Calc. | Obsv. | Calc. | Obsv. | Calc. | Obsv. | Calc. | Obsv. |
| | Mass | Mass | Mass | Mass | Mass | Mass | Mass | Mass |
| Ingredients | (grams) | (grams) | (grams) | (grams) | (grams) | (grams) | (grams) | (grams) |
| 2516 | 4 | 4.2 | 4 | 4.2 | 4 | 4.30 | 4 | 4 |
| Methanol | 1 | 1.1 | 1 | 1.22 | 1 | 1.04 | 1 | 1.66 |
| Glycerol | 0.15 | .20 | | | | | | |
| Propylene | | | 0.15 | 193 | | | | |
| Glycol | | | | | | | | |
| K90 | | | | | 0.15 | .1567 | | |
| Transcutol | | | | | | | 0.5 | .0375 |
| Dapagliflozin | 0.4 | .4097 | 0.4 | .41 | 0.4 | | 0.4 | .4051 |
| Total (g) | 5.55 | 5.90 | 5.55 | 6.023 | 5.55 | 5.89 | 5.9 | 5.66 |

Manufacturing parameters for patches from formulations 8A-8D:
Release liner: 4400
Backing film: 1109
Thickness (drug in adhesive layer coated to the release liner): 0.015 inches
Coating speed: 7 rpm
Drying temperatures: 80° C. (first stage) and 120° C. (second stage)

TABLE 7

| | Obsv. Mass (grams) | | | | | | | | | | |
|---|---|---|---|---|---|---|---|---|---|---|---|
| Ingredients | 10A | 10B | 10C | 10D | 10E | 10F | 10G | 10H | 10I | 10J | 10K |
| 788 | 1.57 | | | | | | | | | | |
| 2852 | | 1.5 | | | | | | | | | |
| 4717A | | | 0.877 | | | | | | | | |
| 4717B | | | 0.775 | | | | | | | | |
| 2516 | | | | 1.52 | 1.68 | 1.59 | 1.62 | 1.51 | 1.53 | 1.5 | 1.69 |
| Ethyl Acetate | 0.375 | 0.376 | | | | | | | | | |
| Methanol | | | | 0.375 | 0.373 | 0.37 | 0.381 | 0.376 | 0.378 | 0.374 | 0.381 |
| Dapagliflozin | 0.187 | 0.1875 | 0.188 | 0.188 | 0.188 | 0.187 | 0.187 | 0.186 | 0.186 | 0.1871 | 0.187 |
| SDS | | | | 0.049 | | | | | | | |
| D-Limonene | | | | | 0.071 | | | | | | |
| DMSO | | | | | | 0.07 | | | | | |
| Tween 20 | | | | | | | 0.071 | | | | |
| K29/32 | | | | | | | | 0.049 | | | |
| Castor Oil | | | | | | | | | 0.05 | | |
| Glycerin | | | | | | | | | | 0.05 | |
| Polyethylene | | | | | | | | | | | 0.058 |
| Glycol | | | | | | | | | | | |
| Total (g) | 2.132 | 2.0635 | 1.84 | 2.132 | 2.312 | 2.217 | 2.259 | 2.121 | 2.144 | 2.1111 | 2.316 |

Manufacturing parameters for patches from formulations 10A-10K:
Release liner: 1022
Backing film: 1109
Thickness (drug in adhesive layer coated to the release liner): 0.015 inches
Coating speed: 7 rpm
Drying temperatures: 80° C. (first stage) and 120° C. (second stage)

TABLE 8

| | | | |
|---|---|---|---|
| 14A | 14B | 14C | 14D |

| | |
|---|---|
| Ingredients | Obsv. Mass (grams) |

| | | | | |
|---|---|---|---|---|
| 2516 | 1.56 | 1.49 | 1.51 | 1.51 |
| Methanol | 0.383 | 0.378 | 0.373 | 0.382 |
| Dapagliflozin | 0.18 | 0.187 | 0.187 | 0.187 |
| VA 64 | 0.05 | | | |
| S-630 | | 0.05 | | |
| K-17 | | | 0.049 | |
| C-30 | | | | 0.049 |
| Total (g) | | | | |

Manufacturing parameters for patches from formulations 14A-14D:
Release liner: 1022
Backing film: 1109
Thickness (drug in adhesive layer coated to the release liner): 0.015 inches
Coating speed: 7 rpm
Drying temperatures: 80° C. (first stage) and 120° C. (second stage)

TABLE 9

| | Obsv. Mass (grams) | | | | | | | | | | | |
|---|---|---|---|---|---|---|---|---|---|---|---|---|
| Ingredients | 18A | 18B | 18C | 18D | 18E | 18F | 18G | 18H | 18I | 18J | 18K | 18L |
| 2516 | 1.52 | 1.55 | 1.54 | 1.52 | 1.54 | 1.51 | 1.64 | 1.52 | 1.57 | 1.53 | 1.54 | 1.5 |
| Methanol | 0.413 | 0.35 | 0.467 | 0.403 | 0.442 | 0.376 | 0.415 | 0.403 | 0.477 | 0.38 | 0.4 | 0.453 |
| Dapagliflozin | 0.187 | 0.186 | 0.188 | 0.187 | 0.187 | 0.186 | 0.187 | 0.187 | 0.187 | 0.189 | 0.186 | 0.186 |
| VA64 | 0.05 | 0.094 | 0.188 | 0.281 | | | | | | | | |
| S-630 | | | | | 0.049 | 0.09 | 0.187 | 0.281 | | | | |
| K-17 | | | | | | | | | 0.05 | 0.09 | 0.18 | 0.28 |
| Total (g) | 2.17 | 2.18 | 2.383 | 2.391 | 2.218 | 2.162 | 2.429 | 2.391 | 2.284 | 2.189 | 2.306 | 2.419 |

Manufacturing parameters for patches from formulations 18A-18L:
Release liner: 1022
Backing film: 1109
Thickness (drug in adhesive layer coated to the release liner): 0.015 inches
Coating speed: 7 rpm
Drying temperatures: 80° C. (first stage) and 120° C. (second stage)

TABLE 10

| | 34A | | 34B | | 34C | | 34D | | 34E | |
|---|---|---|---|---|---|---|---|---|---|---|
| | Calc. | Obsv. | Calc. | Obsv. | Calc. | Obsv. | Calc. | Obsv. | Calc. | Obsv. |
| Ingredients | Mass | Mass | Mass | Mass | Mass | Mass | Mass | Mass | Mass | Mass |
| (% solids) | (g) | (g) | (g) | (g) | (g) | (g) | (g) | (g) | (g) | (g) |
| 2516 (41.5%) | 1 | 2.001 | 1 | 2.17 | 1 | 2.05 | 1 | 2.03 | 1 | 2.05 |
| Methanol | 0.5 | 1 | 0.5 | 1.02 | 0.5 | 0.56 | 0.5 | 1.06 | 0.5 | 1.128 |
| (0%) | | | | | | | | | | |
| Dapagliflozin | 0.15 | 0.1542 | 0.15 | 0.1497 | 0.15 | 0.1532 | 0.15 | 0.1489 | 0.15 | 0.1556 |
| (100%) | | | | | | | | | | |
| Diethylene | 0.0525 | 0.1106 | | | | | | | | |
| Glycol | | | | | | | | | | |
| Monoethyl | | | | | | | | | | |
| Ether | | | | | | | | | | |
| (27.22%) | | | | | | | | | | |
| Propylene | | | 0.0525 | 0.1163 | | | | | | |
| Glycol | | | | | | | | | | |
| (69.5%) | | | | | | | | | | |
| Polysorbate | | | | | 0.0525 | 0.1166 | | | | |
| 80 (97.5%) | | | | | | | | | | |
| Levulinic | | | | | | | 0.0525 | 0.1201 | | |
| Acid | | | | | | | | | | |
| (92.35%) | | | | | | | | | | |
| Lactic Acid | | | | | | | | | 0.0525 | 0.1107 |
| (77.35%) | | | | | | | | | | |
| Total (g) | 1.7025 | 3.2658 | 1.7025 | 3.456 | 1.7025 | 2.8798 | 1.7025 | 3.359 | 1.7025 | 3.4443 |
| Total Solids | | 1.0147 | | 1.1311 | | 1.1176 | | 1.1084 | | 1.092 |
| (g) | | | | | | | | | | |
| % Solids | | 31.07 | | 32.73 | | 38.81 | | 33.00 | | 31.70 |
| % Drug | | 15.2 | | 13.2 | | 13.7 | | 13.4 | | 14.2 |

Manufacturing parameters for patches from formulations 34A-34D:
Release liner: 9744
Backing film: 9738
Thickness (drug in adhesive layer coated to the release liner): 0.015 inches
Coating speed: 7 rpm
Drying temperatures: 80° C. (first stage) and 120° C. (second stage)

TABLE 11

| | 34F | | 34G | | 34H | | 34I | |
|---|---|---|---|---|---|---|---|---|
| | Calc. | Obsv. | Calc. | Obsv. | Calc. | Obsv. | Calc. | Obsv. |
| Ingredients | Mass | Mass | Mass | Mass | Mass | Mass | Mass | Mass |
| (% solids) | (g) | (g) | (g) | (g) | (g) | (g) | (g) | (g) |
| 2516 (41.5%) | 1 | 1.03 | 1 | 2.04 | 1 | 2.5 | 1 | 2.29 |
| Methanol | 0.5 | 0.5616 | 0.5 | 0.5706 | 0.5 | 0.5582 | 0.5 | 1.11 |
| (100%) | | | | | | | | |
| Dapagliflozin | 0.15 | 0.1584 | 0.15 | 0.1525 | 0.15 | 0.143 | 0.15 | 0.1557 |
| (100%) | | | | | | | | |
| Crodamol | 0.0525 | 0.11 | | | | | | |
| (99.47%) | | | | | | | | |
| Eucalyptus | | | 0.0525 | 0.113 | | | | |
| Oil (0.04%) | | | | | | | | |
| Prosolv 730 | | | | | 0.0525 | 0.1 | | |
| (100%) | | | | | | | | |
| Plastoid B | | | | | | | 0.0525 | 0.2 |
| (100%) | | | | | | | | |
| Total (g) | 1.7025 | 1.86 | 1.7025 | 2.8761 | 1.7025 | 3.3012 | 1.7025 | 3.7557 |
| Total Solids (g) | | 0.6953 | | 0.99914 | | 1.2805 | | 1.3061 |
| % Solids | | 37.38 | | 34.74 | | 38.79 | | 34.78 |
| % Drug | | 22.8 | | 15.3 | | 11.2 | | 11.9 |

Manufacturing parameters for patches from formulations 34F-34I:
Release liner: 9744
Backing film: 9738
Thickness (drug in adhesive layer coated to the release liner): 0.015 inches
Coating speed: 7 rpm
Drying temperatures: 80° C. (first stage) and 120° C. (second stage)

TABLE 12

| | 34J | | 34K | | 34L | | 34M | | 34N | |
|---|---|---|---|---|---|---|---|---|---|---|
| | Calc. | Obsv. | Calc. | Obsv. | Calc. | Obsv. | Calc. | Obsv. | Calc. | Obsv. |
| | Mass | Mass | Mass | Mass | Mass | Mass | Mass | Mass | Mass | Mass |
| Ingredients | (g) | (g) | (g) | (g) | (g) | (g) | (g) | (g) | (g) | (g) |
| 2516 | 1 | 2.036 | 1 | 3.28 | 1 | 2.75 | 1 | 2.55 | 1 | 2.11 |
| Methanol | 0.5 | 1.0923 | 0.5 | 1.11 | 0.5 | 0.5757 | 0.5 | 0.562 | 0.5 | 0.6 |
| Dapagliflozin | 0.15 | 0.152 | 0.15 | 0.1519 | 0.15 | 0.1554 | 0.15 | 0.158 | 0.15 | 0.1572 |
| Kollidon CL | 0.0525 | 0.1233 | | | | | | | | |
| PVP | | | 0.0525 | 0.189 | | | | | | |
| avg. mol. wt | | | | | | | | | | |
| 40,000 | | | | | | | | | | |
| K-17 | | | | | 0.0525 | 0.0978 | | | | |
| K-29/32 | | | | | | | 0.0525 | 0.905 | | |
| Polyplasdone XL | | | | | | | | | 0.0525 | 0.78 |
| Total (g) | 1.7025 | 3.4036 | 1.7025 | 4.7309 | 1.7025 | 3.5789 | 1.7025 | 4.175 | 1.7025 | 3.6472 |
| Total Solids (g) | | 1.1202 | | 1.1858 | | 1.7894 | | 2.1213 | | 2.6885 |
| % Solids | | 32.91 | | 25.07 | 0.00 | 50.00 | | 50.81 | | 73.71 |
| % Drug | | 13.6 | | 12.8 | | 8.7 | | 7.4 | | 5.8 |

Manufacturing parameters for patches from formulations 34J-34N:
Release liner: 9744
Backing film: 9738
Thickness (drug in adhesive layer coated to the release liner): 0.015 inches
Coating speed: 7 rpm
Drying temperatures: 80° C. (first stage) and 120° C. (second stage)

TABLE 13

| | 39B | | | 39C | | | 39D | | |
|---|---|---|---|---|---|---|---|---|---|
| | Calc. | Obsv. | | Calc. | Obsv. | | Calc. | Obsv. | |
| Ingredients | Mass | Mass | % as is | Mass | Mass | % as is | Mass | Mass | % as is |
| (% solids) | (g) | (g) | (w/w) | (g) | (g) | (w/w) | (g) | (g) | (w/w) |
| 2516 | 4 | 4 | 62.5 | 4 | 4.03 | 60.61 | 4 | 4.0328 | 59.52 |
| (41.5%) | | | | | | | | | |
| Methanol | 2 | 2.02 | 31.25 | 2 | 2.05 | 30.30 | 2.12 | 2.12 | 31.55 |
| Dapagliflozin | 0.40 | 0.4 | 6.25 | 0.40 | 0.401 | 6.06 | 0.40 | 0.4001 | 5.95 |
| (100%) | | | | | | | | | |
| Polysorbate | | | | 0.20 | 0.2117 | 3.03 | | | |
| 80 | | | | | | | | | |
| (97.89% | | | | | | | | | |
| solids) | | | | | | | | | |
| Levulinic | | | | | | | 0.20 | 0.2367 | 2.98 |
| Acid | | | | | | | | | |
| (94.70% | | | | | | | | | |
| solids) | | | | | | | | | |
| Total (g) | 6.4 | 6.42 | | 6.6 | 6.6927 | | 6.72 | 6.7896 | |
| Total Solids (g) | | 2.06 | | | 2.28 | | | 2.29 | |
| % Solids | | 32.09 | | | 34.07 | | | 33.73 | |
| % Drug | | 19.42 | | | 17.59 | | | 17.47 | |

Manufacturing parameters for patches from formulations 39B-39D:
Release liner: 9744
Backing film: 9720
Thickness (drug in adhesive layer coated to the release liner): 0.015 inches
Coating speed: 7 rpm
Drying temperatures: 80° C. (first stage) and 120° C. (second stage)

TABLE 14

| | 51A | |
|---|---|---|
| Ingredients (% solids) | Calc. Mass (g) | Obsv. Mass (g) |
| 2516 (41.5%) | 4 | 4.04 |
| Methanol | 2 | 2.03 |
| Dapagliflozin (100%) | 0.40 | 0.4278 |
| Levulinic Acid (94.70% solids) | 0.2 | 0.2226 |

Manufacturing parameters for patches from formulation 51A:
Release liner: 9744
Backing film: 9700, 9701, or 9720
Thickness (drug in adhesive layer coated to the release liner): 0.016 inches
Coating speed: 6 rpm
Drying temperatures: 80° C. (first stage) and 120° C. (second stage)

### Example 3: Skin Permeation Analysis of Dapagliflozin TDS Patches

Performance of the formulations was evaluated based on skin permeation (TABLES 15-18). Skin permeation studies were conducted with a Franz cell setup using human cadaver skin.

The skin permeation apparatus used thermo heat units with aluminum blocks milled out to accept standard Franz cells. Each unit was equipped with temperature and stirring controls. Independent temperature calibration probes were used to verify the temperature of each block. Sodium phosphate buffer solution was used in the Franz cell. The general procedure for skin permeation experiments was as follows:
1. Turn on the thermo temperature units to 37° C.+/−5° C.
2. Prepare the components of the Franz cell
   a. Rinse the Franz cell reservoir with deionized water, clean, and dry before use
   b. Thaw the skin sample to be used
   c. Cut out a circular sample from the skin that is ⅝ inch in diameter
   d. Cut out a circular sample of the transdermal patch to be tested that is ⅜ inch in diameter
   e. Cut out a circular sample of the overlay (foam tape or the equivalent) that is 1 inch in diameter
3. Assemble the Franz cell
   a. Remove the release liner from the transdermal patch. Place the patch in the center of the circular sample of skin on the epidermis. Ensure that the skin is dry and the patch is placed on the correct side of the skin
   b. Remove the release liner from the overlay and place the release liner on top of the skin sample with the patch. Carefully align the patch with the circle opening of the Franz cell and ensure the Franz cell is tightly wrapped in Parafilm™
   c. Once the Franz cell is assembled, fill the cell with sodium phosphate buffer (pH 7.2), ensuring that all bubbles are removed from the cell. Insert the stir bar into the Franz cell
   d. Fill a container with sodium phosphate buffer solution and maintain the buffer at the same temperature as the Franz cells
4. Insert the assembled Franz cell into the thermo unit
5. Set the thermo unit to maintain a temperature of 32° C.+/−. 5° C.
6. Turn on the stir bar mixer to high speed
7. Sample the Franz cell at prescribed time intervals
   a. Using a disposable pipette, insert the pipette into the sampling arm. Fill the pipette and dispose of the first draw
   b. Fill the pipette a second time and use the second draw as the sample to be tested
   c. Empty the contents of the Franz cell and refill using sodium phosphate buffer

TABLE 15

| | | | Average | | |
|---|---|---|---|---|---|
| | | Dapagliflozin | Dapagliflozin | Dapagliflozin | Average |
| | | Delivered | Delivered | Delivered | Dapagliflozin |
| | Dapagliflozin | (mg) from | (mg) from | (%) from | Delivered (%) |
| | (mg) in 0.497 | 0.497 cm² | 0.497 cm² | 0.497 cm² | from 0.497 |
| Formulation | cm² patch | patch | patch | patch | cm² patch |
| 3A - 1 | 1.11 | 0.13 | 0.24 | 11.76 | 23.48 |
| 3A - 2 | 1.02 | 0.36 | | 35.21 | |
| 3B - 1 | 0.97 | 0.51 | 0.64 | 52.35 | 65.53 |
| 3B - 2 | 0.99 | 0.78 | | 78.71 | |
| 3B - 3 | 1.32 | 0.44 | 0.34 | 33.60 | 23.41 |
| 3B - 4 | 1.23 | 0.17 | | 14.01 | |
| 3B - 5 | 1.75 | 0.39 | | 22.61 | |
| 3B - 6 | 0.97 | 0.01 | 0.01 | 1.09 | 1.18 |
| 3B - 7 | 0.97 | 0.01 | | 1.28 | |
| 3C - 1 | 1.00 | 0.12 | 0.07 | 12.23 | 7.01 |
| 3C - 2 | 1.05 | 0.02 | | 1.79 | |
| 7A - 1 | 1.27 | 0.09 | 0.17 | 6.71 | 12.785 |
| 7A - 2 | 1.37 | 0.26 | | 18.86 | |
| 7B - 1 | 1.00 | 0.01 | 0.02 | 1.07 | 1.58 |
| 7B - 2 | 0.93 | 0.02 | | 2.09 | |
| 7C - 1 | 0.71 | 0.02 | 0.02 | 2.67 | 3.335 |
| 7C - 2 | 0.73 | 0.03 | | 4.00 | |
| 7D - 1 | 1.10 | 0.01 | 0.01 | 0.79 | .725 |
| 7D - 2 | 1.03 | 0.01 | | 0.66 | |
| 8A - 1 | 0.96 | 0.03 | 0.90 | 2.63 | 5.77 |
| 8A - 2 | 0.84 | 0.07 | | 8.91 | |
| 8B - 1 | 0.88 | 0.09 | 1.00 | 10.26 | 10.60 |
| 8B - 2 | 1.13 | 0.12 | | 10.93 | |
| 8B - 3 | 0.93 | 0.02 | 0.02 | 1.63 | 1.83 |
| 8B - 4 | 0.93 | 0.02 | | 2.04 | |
| 8C - 1 | 0.89 | 0.14 | 0.89 | 16.25 | 14.65 |
| 8C - 2 | 0.89 | 0.12 | | 13.05 | |
| 8C - 3 | 0.87 | 0.01 | 0.01 | 1.30 | 1.46 |
| 8C - 4 | 0.87 | 0.01 | | 1.62 | |
| 8D - 1 | 0.96 | 0.06 | 0.94 | 6.55 | 4.28 |
| 8D - 2 | 0.91 | 0.02 | | 2.01 | |
| 10A - 1 | 0.98112 | 0.059 | 0.040 | 6.02 | 4.39 |
| 10A - 2 | 0.77088 | 0.021 | | 2.77 | |
| 10B - 1 | 0.92378 | 0.021 | 0.021 | 2.28 | 2.44 |
| 10B - 2 | 0.8151 | 0.021 | | 2.60 | |
| 10C - 1 | 1.21552 | 0.014 | 0.011 | 1.12 | 0.94 |
| 10C - 2 | 1.17008 | 0.009 | | 0.76 | |
| 10D - 1 | 1.09 | 0.023 | 0.031 | 2.16 | 2.61 |
| 10D - 2 | 1.0246 | 0.031 | | 3.06 | |
| 10E - 1 | 0.97196 | 0.015 | 0.016 | 1.54 | 1.66 |
| 10E - 2 | 0.94987 | 0.017 | | 1.79 | |
| 10F - 1 | 1.1365 | 0.039 | 0.029 | 3.42 | 2.54 |
| 10F - 2 | 1.15923 | 0.019 | | 1.66 | |
| 10G - 1 | 0.93869 | 0.021 | 0.017 | 2.20 | 1.77 |
| 10G - 2 | 0.98235 | 0.013 | | 1.33 | |
| 14A - 1 | 0.981 | 0.15 | 0.14 | 14.96 | 14.29 |
| 14A - 2 | 0.9374 | 0.13 | | 13.63 | |
| 14B - 1 | 0.9592 | 0.21 | 0.22 | 21.51 | 23.07 |
| 14B - 2 | 0.981 | 0.24 | | 24.64 | |
| 14B - 3 | 0.92 | 0.01 | 0.02 | 1.63 | 2.32 |
| 14B - 4 | 0.92 | 0.03 | | 3.00 | |
| 14C - 1 | 0.872 | 0.28 | 0.21 | 32.46 | 23.34 |
| 14C - 2 | 0.9374 | 0.13 | | 14.22 | |
| 14C - 3 | 0.92 | 0.02 | 0.02 | 2.68 | 2.29 |
| 14C - 4 | 0.92 | 0.02 | | 1.91 | |
| 14D - 1 | 0.8502 | 0.13 | 0.10 | 15.81 | 12.79 |
| 14D - 2 | 0.7412 | 0.07 | | 9.77 | |
| 18A - 1 | 1.13 | 0.06 | 0.10 | 5.23 | 8.77 |
| 18A - 2 | 1.20 | 0.15 | | 12.31 | |
| 18B - 1 | 1.06 | 0.09 | 0.12 | 8.34 | 9.34 |
| 18B - 2 | 1.47 | 0.15 | | 10.35 | |
| 18C - 1 | 1.24 | 0.07 | 0.06 | 5.97 | 4.87 |
| 18C - 2 | 1.18 | 0.04 | | 3.69 | |
| 18D - 1 | 1.37 | 0.08 | 0.07 | 5.67 | 5.86 |
| 18D - 2 | 1.12 | 0.07 | | 6.05 | |
| 18E - 1 | 1.20 | 0.07 | 0.08 | 5.86 | 6.62 |
| 18E - 2 | 1.26 | 0.09 | | 7.39 | |
| 18F - 1 | 1.26 | 0.06 | 0.10 | 4.65 | 7.64 |
| 18F - 2 | 1.39 | 0.15 | | 10.63 | |
| 18G - 1 | 1.40 | 0.02 | 0.02 | 1.33 | 1.79 |
| 18G - 2 | 1.22 | 0.03 | | 2.25 | |
| 18G - 3 | 1.08 | 0.01 | 0.01 | 1.09 | 1.43 |
| 18G - 4 | 1.08 | 0.01 | | 1.77 | |
| 18H - 1 | 1.32 | 0.03 | 0.03 | 1.98 | 1.96 |
| 18H - 2 | 1.49 | 0.03 | | 1.95 | |
| 18I - 1 | 1.66 | 0.06 | 0.06 | 3.84 | 3.67 |
| 18I - 2 | 1.35 | 0.05 | | 3.50 | |
| 18J - 1 | 1.68 | 0.05 | 0.05 | 2.80 | 3.55 |
| 18J - 2 | 1.26 | 0.05 | | 4.31 | |
| 18J - 3 | 1.12 | 0.01 | 0.01 | 1.12 | 1.15 |
| 18J - 4 | 1.12 | 0.01 | | 1.18 | |
| 23A - 1 | 0.68 | 0.05 | 0.04 | 7.17 | 5.80 |
| 23A - 2 | 0.68 | 0.03 | | 4.43 | |
| 23B - 1 | 0.61 | 0.04 | 0.04 | 6.30 | 6.64 |
| 23B - 2 | 0.61 | 0.04 | | 6.98 | |
| 23C - 1 | 0.26 | 0.04 | 0.03 | 16.29 | 13.24 |
| 23C - 2 | 0.26 | 0.03 | | 10.19 | |

TABLE 16

| | | | | Estimated | | |
|---|---|---|---|---|---|---|
| | | | | Average | | Average |
| | | | Average | Dapaglifloz | Dapaglifloz | Dapaglifloz |
| | | Dapaglifloz | Dapaglifloz | in | in | in |
| | Dapaglifloz | in Delivered | in Delivered | Delivered | Delivered | Delivered |
| | in (mg) in | (mg) from | (mg) | (mg) from | (%) from | (%) from |
| | 0.497 cm² | 0.497 cm² | from0 .497 | 25 cm² | 0.497 cm² | 0.497 cm² |
| Formulation | patch | patch | cm² patch | patch | patch | patch |
| 34N-1 | 0.01 | 0.06 | 0.065 | 3.30 | 410.71 | 278.48 |
| 34N-2 | 0.02 | 0.07 | | | 416.19 | |
| 34D-1 | 0.36 | 0.33 | 0.28 | 13.05 | 91.83 | 66.12 |
| 34D-2 | 0.38 | 0.23 | | | 62.02 | |
| 34C-1 | 0.40 | 0.10 | 0.115 | 9.58 | 26.11 | 52.20 |
| 34C-2 | 0.33 | 0.13 | | | 38.66 | |
| 3B-1 | 0.70 | 0.06 | 0.055 | 2.92 | 8.55 | 36.22 |
| 3B-2 | 0.66 | 0.05 | | | 8.28 | |
| 34E-1 | 0.45 | 0.20 | 0.185 | 8.55 | 44.51 | 32.80 |
| 34E-2 | 0.41 | 0.17 | | | 41.59 | |
| 34B-1 | 0.26 | 0.08 | 0.085 | 4.65 | 28.66 | 25.05 |
| 34B-2 | 0.46 | 0.09 | | | 20.40 | |
| 34J-1 | 0.46 | 0.18 | 0.135 | 5.92 | 39.67 | 24.22 |
| 34J-2 | 0.52 | 0.09 | | | 17.88 | |
| 34M-1 | 0.22 | 0.07 | 0.055 | 2.89 | 29.95 | 23.71 |
| 34M-2 | 0.25 | 0.04 | | | 17.46 | |
| 341-1 | 0.57 | 0.04 | 0.045 | 4.62 | 6.42 | 19.00 |
| 341-2 | 0.46 | 0.05 | | | 10.91 | |
| 34G-1 | 0.57 | 0.15 | 0.14 | 5.26 | 25.62 | 18.47 |
| 34G-2 | 0.58 | 0.13 | | | 21.66 | |
| 34L-1 | 0.35 | 0.05 | 0.045 | 2.63 | 13.44 | 18.36 |
| 34L-2 | 0.36 | 0.04 | | | 11.67 | |
| 34A-1 | 0.57 | 0.06 | 0.06 | 3.42 | 11.15 | 16.90 |
| 34A-2 | 0.57 | 0.06 | | | 10.89 | |
| 34F-1 | 1.05 | 0.13 | 0.095 | 5.63 | 12.30 | 14.46 |
| 34F-2 | 1.05 | 0.06 | | | 5.45 | |
| 34K-1 | 0.47 | 0.07 | 0.055 | 2.77 | 15.12 | 13.15 |
| 34K-2 | 0.41 | 0.04 | | | 10.88 | |
| 34H-1 | 0.47 | 0.04 | 0.05 | 2.24 | 8.13 | 9.68 |
| 34H-2 | 0.39 | 0.06 | | | 14.49 | |

TABLE 17

| | Average Dapagliflozin | Average Dapagliflozin |
|---|---|---|
| | Delivered (mg) from 0.497 | Delivered (mg) from 25 |
| Formulation | cm² samples after 7 days | cm² samples after 7 days |
| 3B - 1 | 0.45 (stratum corneum) | 23.06 (stratum corneum) |
| 3B - 2 | 0.16 (500 μM back) | 8.20 (500 μM back) |
| 8C - 1 | 0.52 (stratum corneum) | 26.58 (stratum corneum) |
| 8C - 2 | 0.08 (500 μM back) | 3.97 (500 μM back) |
| 14B - 1 | 0.31 (stratum corneum) | 15.76 (stratum corneum) |
| 14B - 2 | 0.12 (500 μM back) | 6.10 (500 μM back) |
| 14C - 1 | 0.36 (stratum corneum) | 18.14 (stratum corneum) |
| 14C - 2 | 0.12 (500 μM back) | 6.29 (500 μM back) |
| 23A - 1 | 0.04 (stratum corneum) | 2.11 (stratum corneum) |
| 23A - 2 | 0.03 (500 μM back) | 1.59 (500 μM back) |
| 23B - 1 | 0.03 (stratum corneum) | 1.68 (stratum corneum) |
| 23B - 2 | 0.04 (500 μM back) | 1.80 (500 μM back) |

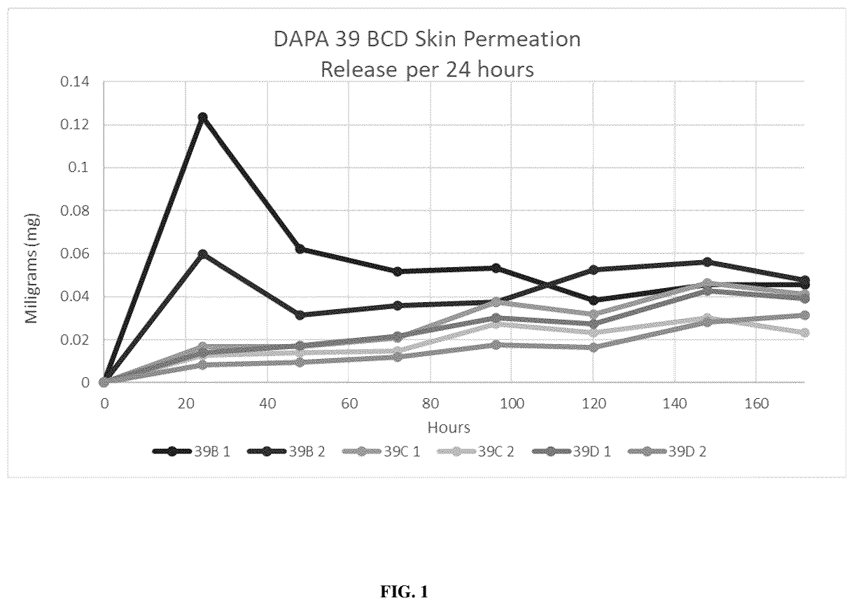

TDS patches containing dapagliflozin were punched out into 0.497 cm² circular samples for testing in Formulations 39B, 39C, and 39D. Skin permeation results are shown in TABLE 18 and FIGS. 1-8. For Formulations 39B, 39C, and 39D, methanol can be varied by +/−5% and the adhesive composition can be varied by +/−10% with no effect on the final product. Enhancers can tolerate a +/−5% variation.

TABLE 18

| | | | Average | | |
|---|---|---|---|---|---|
| | | Dapagliflozin | Dapagliflozin | Dapagliflozin | Average |
| | | Delivered | Delivered | Delivered | Dapagliflozin |
| | Dapagliflozin | (mg) from | (mg) from | (%) from | Delivered (%) |
| | (mg) in 0.497 | 0.497 cm² | 0.497 cm² | 0.497 cm² | from 0.497 |
| Formulation | cm² patch | patch | patch | patch | cm² patch |
| 39B - 1 | 0.23 | 0.420 | 0.370 | 182.70* | 164.10 |
| 39B - 2 | 0.22 | 0.320 | | 145.49* | |
| 39C - 1 | 0.23 | 0.211 | 0.178 | 93.90 | 75.18 |
| 39C - 2 | 0.26 | 0.146 | | 56.45 | |
| 39D - 1 | 0.23 | 0.192 | 0.158 | 83.07 | 68.35 |
| 39D - 2 | 0.23 | 0.124 | | 53.64 | |

*% delivery of 39B > 100% - can be associated with microtears in the skin during the preparatory phase of skin permeation.

FIG. 1 provides the dapagliflozin delivery (mg) from formulations 39B, 39C, and 39D over 172 hours from skin permeation studies, plotted as delivery every 24 hours.

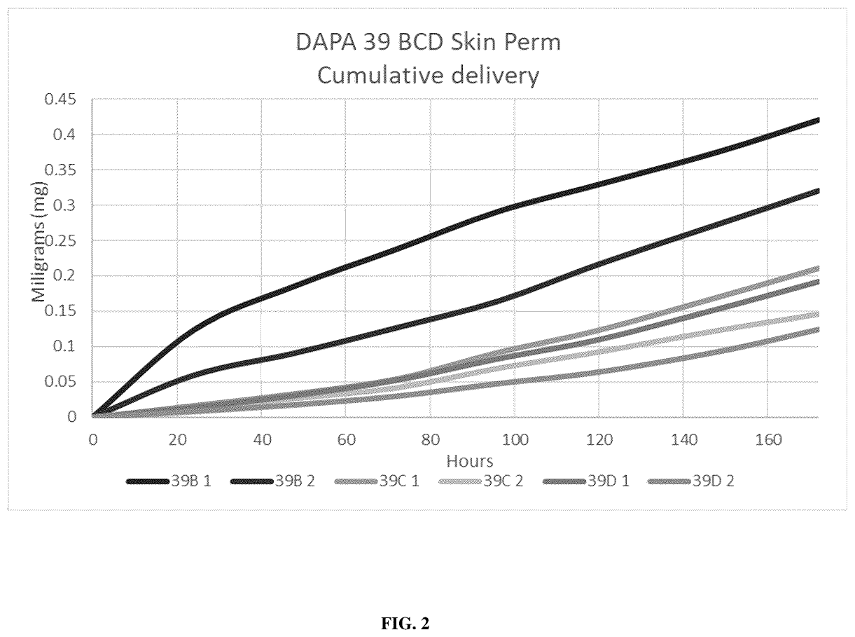

FIG. 2 provides the dapagliflozin delivery (mg) from formulations 39B, 39C, and 39D over 172 hours from skin permeation studies, plotted as cumulative delivery.

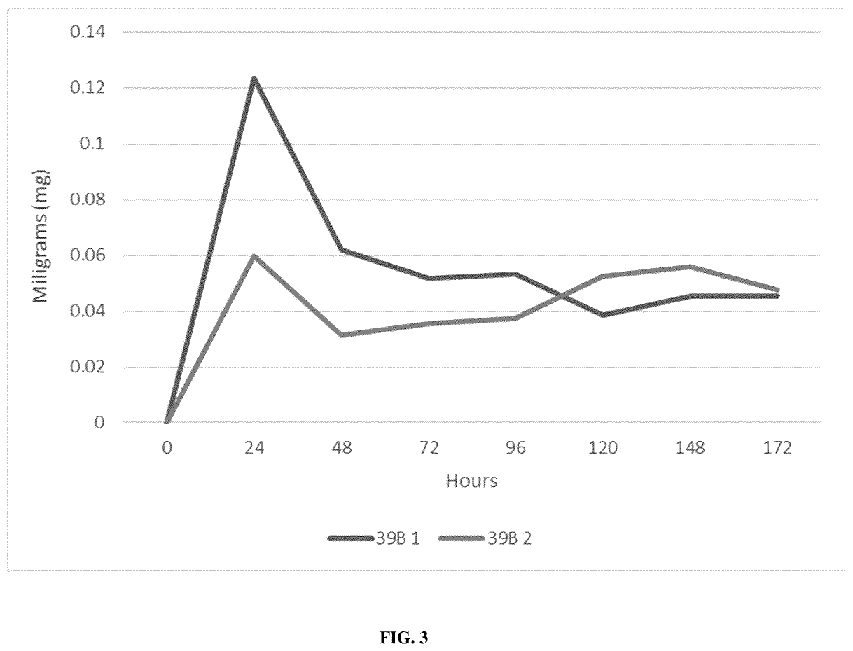

FIG. 3 provides the dapagliflozin delivery (mg) from formulation 39B over 172 hours from skin permeation studies, plotted as delivery every 24 hours.

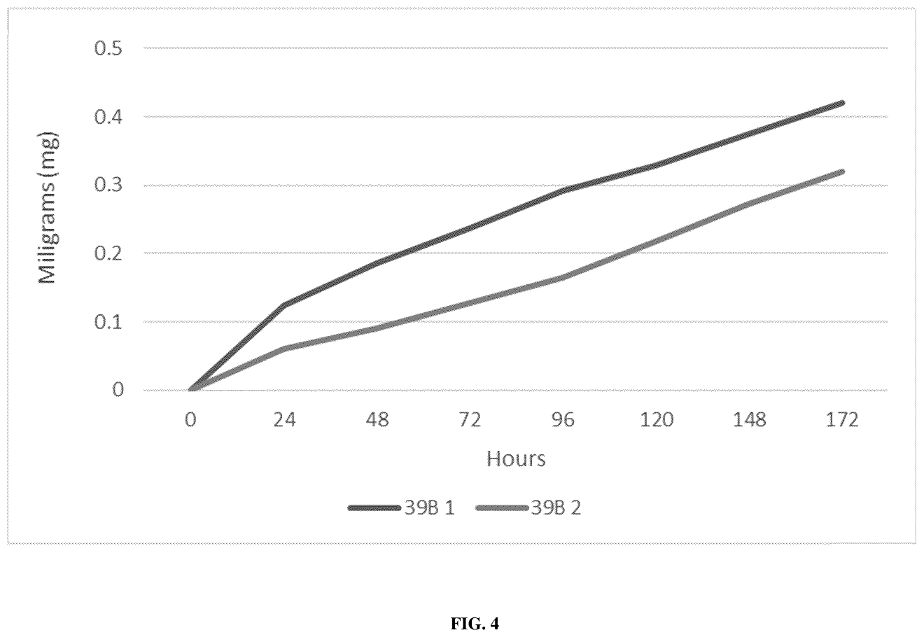

FIG. 4 provides the dapagliflozin delivery (mg) from formulation 39B over 172 hours from skin permeation studies, plotted as cumulative delivery.

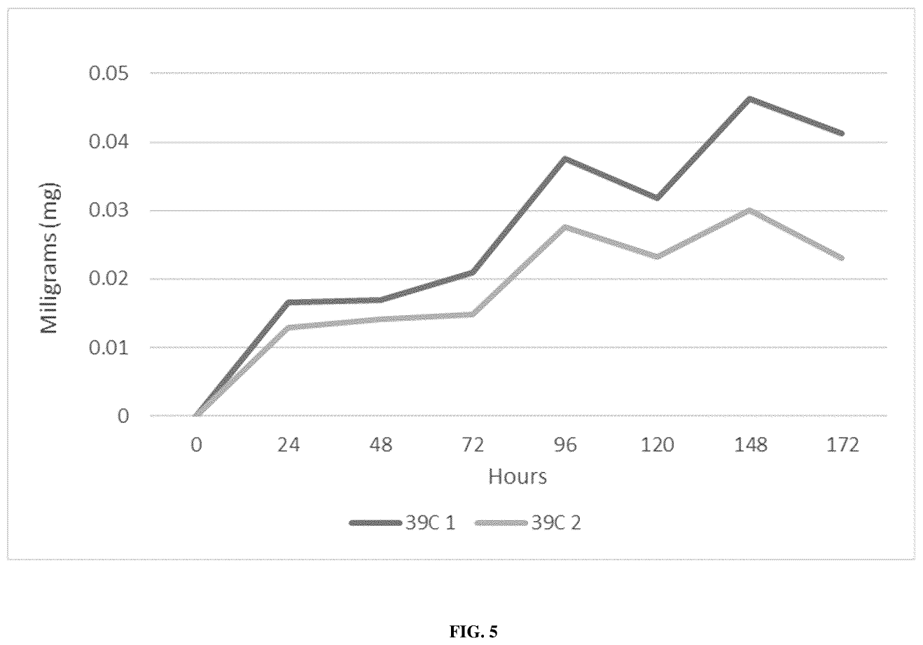

FIG. 5 provides the dapagliflozin delivery (mg) from formulation 39C over 172 hours from skin permeation studies, plotted as delivery every 24 hours.

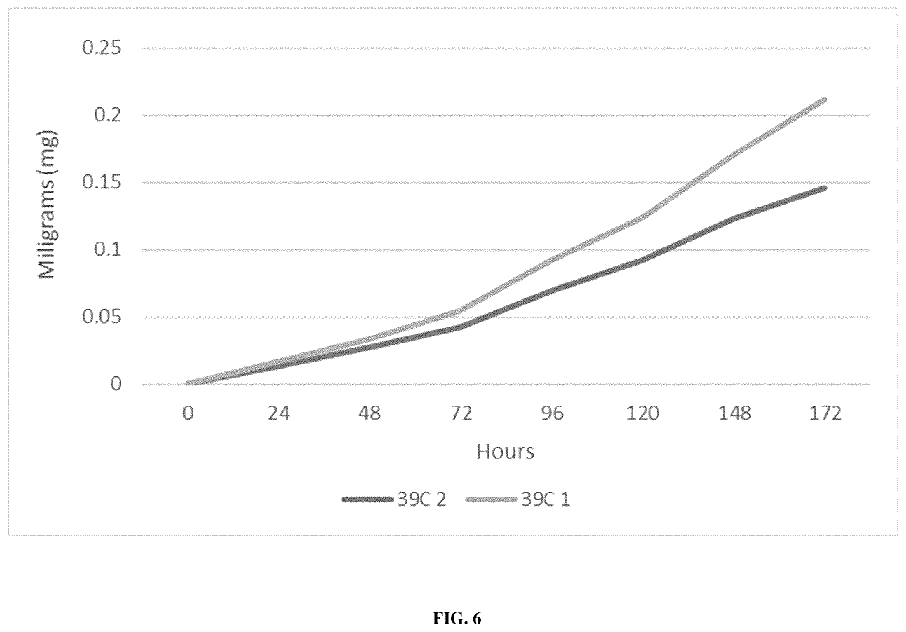

FIG. 6 provides the dapagliflozin delivery (mg) from formulation 39C over 172 hours from skin permeation studies, plotted as cumulative delivery.

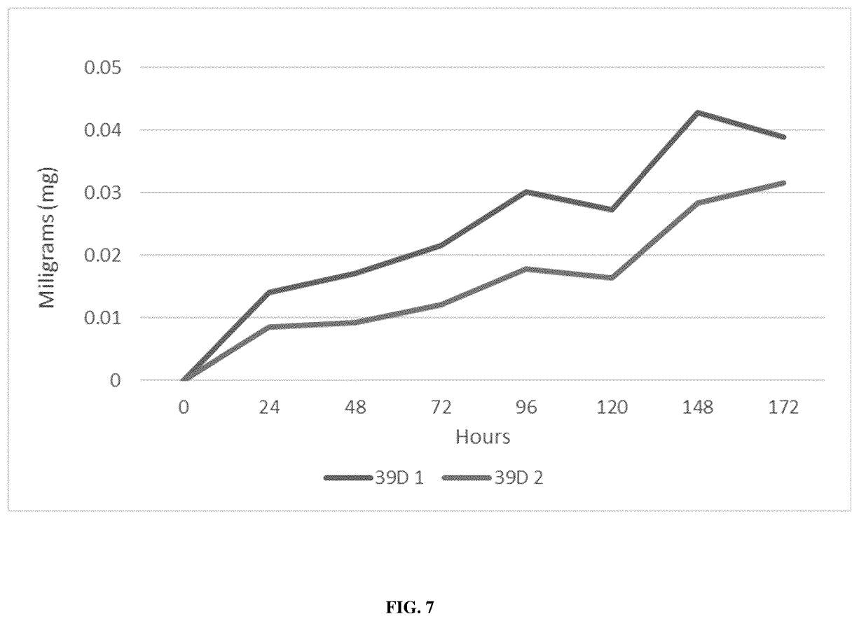

FIG. 7 provides the dapagliflozin delivery (mg) from formulation 39D over 172 hours from skin permeation studies, plotted as delivery every 24 hours.

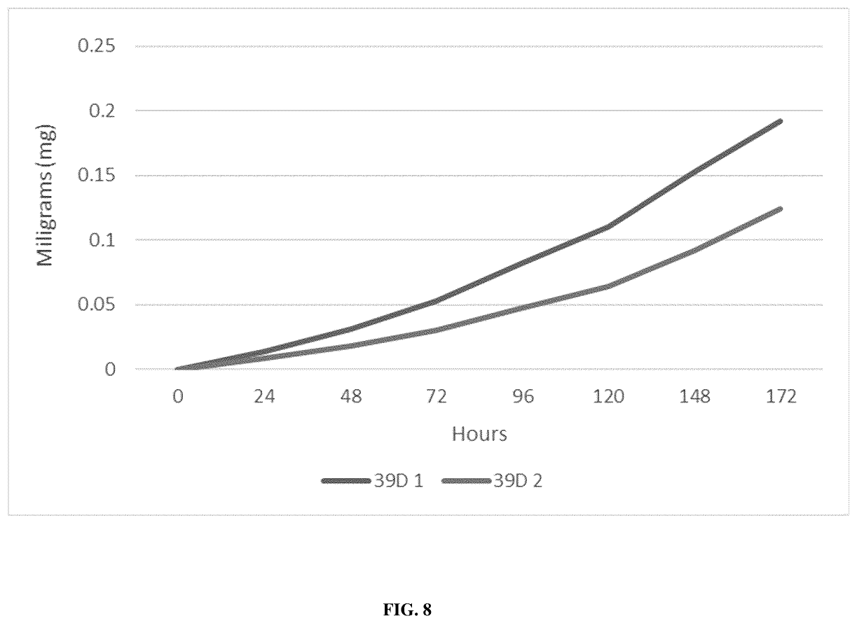

FIG. 8 provides the dapagliflozin delivery (mg) from formulation 39D over 172 hours from skin permeation studies, plotted as cumulative delivery.

### Example 4: Tack, Shear, and Peel Properties of Dapagliflozin and Placebo TDS Patches

Tack, shear, and peel testing of dapagliflozin and placebo TDS patches were carried out according to the general procedures below. TABLES 19-23 document the tack, shear, and peel properties of select formulations.

Tack testing was performed to determine the force and time required to break the adhesive bond from a test probe. In this experiment, for a 0.5 cm² sample, an effective pressure (the pull force on the sample) is above 7 grams and the ideal time is around 5 minutes to result in high tack upon initial application of the patch. Tack testing was performed using a HMP model 2300 pull tester having a test plate, pressure gauge, ¼ and ½ inch probes, and a built-in damper. The general procedure for tack testing is as follows:
1. Insert the horizontal plate on the tester
2. Cut a roughly ¾ inch wide by 2 inch-long sample from the patch to be tested
3. Peel off the release liner and place the backing film on the surface of the test plate, using tape to stabilize both edges of the patch to the surface
4. Using either the ¼ inch or ½ inch metal cylinder probe, place the probe on the plastic tip of the instrument
5. Adjust the gram meter black arm to read 0
6. Slowly turn the gram meter knob so the probe lowers until touching the patch. Slowly let go of the knob and start the timer
7. Record the force in grams applied from the black dial indicator
8. Record the time in seconds taken for the probe to release from the patch.
9. Document any observations regarding physical release
10. Clean the probe with ethyl acetate between use and let dry before conducting the next test

Shear testing was performed to determine the maximum pressure needed for the sample to start shearing from the test plate. The sample was applied to the test plate and the other end was clamped to a motorized pull gauge, which applied continuous force that was read out by a test gauge. A sample with over 500 grams resistance was tested to last up to 7 days with little to no lifting. Shear testing was performed using a HMP Model 1850 Lead Pull Tester having a rotatable test plate and a one-pound roller to secure the sample on the test plate. The general procedure for shear testing was as follows:
1. Cut out a ½ inch wide by 1.5-2 inch long sample from the patch to be tested
2. Peel off the release liner exposing the formulation side
3. Place the adhesive side of the patch to touch the test plate within the engraved area
4. Using a one-pound steel roller, roll over the test piece three times applying no other pressure except for the weight of the roller
5. Insert the test plate perpendicular to the instrument clamp
6. Turn on the instrument. Hit the reset button to have the unit move forward to the test piece
7. Insert the uncoated liner into the clamp and tighten. Ensure that the sample is taught
8. Reset the pressure gauge to 0
9. Start the test and run the sample until the machine stops
10. Record the maximum pressure applied in ounces from the test gauge. Convert ounces to grams based on 0.1 cm² sample surface area
11. Document any observations regarding physical release
12. Clean the test plate with ethyl acetate between use and let dry before conducting the next test
13. Conduct the test a second time and record pressure
14. Calculate the average pressure between two runs and record the average as the final result. Duplicates are within +/−20% for pressure

Peel testing was performed to determine the maximum pressure needed for the sample to start pulling from the test plate at a 90° or 180° angle. The sample is applied to the test plate and the other end is clamped to a motorized pull gauge, which applies continuous force that is read out by a test gauge. In this experiment, a desirable release is around 300 grams per 5 cm² sample; pressure above 400 grams can translate to the patch starting to pull hair from skin and causing irritation upon removal. Peel testing is performed using a HMP Model 1850 Lead Pull Tester having a rotatable test plate and a one-pound roller to secure the sample on the test plate. The general procedure for peel testing is as follows:
1. Cut out a ½ inch wide by 1.5-2 inch long sample from the patch to be tested
2. Peel off the release liner to expose the formulation side
3. Place the adhesive side of the patch to touch the test plate within the engraved area, ensuring that the sample edge is at the edge of the test plate
4. Using a one-pound steel roller, roll over the sample three times applying no other pressure except the weight of the roller. Once pressure is applied, slowly pull the sample until the sample reaches the engrave line on the test plate
5. Insert the test plate and adjust the plate to be 90° or 180° to the instrument clamp
6. Turn on the instrument. Hit the reset button to have the unit move forward to the test piece
7. Insert the uncoated liner into the clamp and tighten. Ensure the sample is taught
8. Reset the pressure gauge to 0
9. Start the test and run the sample until the machine stops
10. Record the maximum pressure applied in ounces from the test gauge. Convert ounces to grams
11. Document any observations regarding physical release
12. Clean the test plate with ethyl acetate between use and let dry before conducting the next test
13. Conduct the test a second time and record pressure
14. Calculate the average pressure between two runs and record the average as the final result. Duplicates are within +/−20% for pressure

TABLE 19

| Formu- | Tack Testing | Tack Testing | Peel Testing | Peel testing |
|---|---|---|---|---|
| lation | Pressure (g) | Time (s) | 90° (oz) | 180° (g) |
| 3A | 8 | 13 | 0 | 0 |
| 3B | 8 | 9 | 15 | 425 |
| 3C | 7 | >5 min | 5 | 142 |

TABLE 20

| | Tack Testing | Tack Testing |
|---|---|---|
| Sample | Average Time (minutes) | Pressure (grams) |
| 3B Placebo | 5 | 7 |
| 34C Placebo | 0.45 | 7.5 |
| 34D Placebo | 5 | 7 |
| 39D Placebo | 5 | 7 |
| 39C Placebo | 5 | 7 |
| 34D | 5 | 7 |
| 39D | 5 | 7 |
| 39C | 5 | 7.5 |
| 39B | 5 | 7.5 |
| 34M | 5 | 7.5 |
| 34N | 2 | 7.5 |
| 34L | 5 | 7.5 |

TABLE 21

| | Shear Testing |
|---|---|
| Sample | Pressure (grams) |
| 3B | 680 |
| 34C | 595 |
| 34D | 680 |
| 39D | 623 |
| 34L | 680 |
| 34M | 538 |
| 34N | 680 |
| 39D Placebo | 652 |
| 34D Placebo | 680 |

TABLE 22

| | 180 Degree Peel |
|---|---|
| Sample | Pressure (grams) |
| 3B | 566 |
| 34C Placebo | 56 |
| 34D Placebo | 170 |
| 39D Placebo | 155 |
| 34D | 396 |
| 34M | 467 |
| 34N | 461 |
| 34L | 226 |
| 39B | 354 |
| 39C | 297 |
| 39D | 297 |

TABLE 23

| | 90 Degree Peel |
|---|---|
| Sample | Pressure (grams) |
| 39B | 297 |
| 39C | 311 |
| 39D | 340 |

### Example 5: Dissolution Properties of Dapagliflozin TDS Patches

Dissolution studies were performed to evaluate the release rates of dapagliflozin from TDS patches over time. These studies were performed using a Distek Model 2100 instrument with a programmable sampler, heating unit, and dissolution bath containing 6 vessels. The samples were inserted into a sunken basket with a rotating paddle overhead. Samples were analyzed up to 172 hours to determine the long-term ability of the patch to deliver dapagliflozin. TABLE 24 shows the conversion table used to establish area for TDS calculations.

TABLE 24

| Diameter (in) | Circumference (in) | Area (in²) | Area (cm²) |
|---|---|---|---|
| 5/16 | 0.982 | 0.077 | 0.497 |
| 5/8 | 1.96 | 0.31 | 1.355 |

The general procedure for dissolution testing is as follows:
1. Ensure that the temperature controller is calibrated
2. Ensure that the autosampler is calibrated with the instrument set up to collect 2 mL of sample at each time point
3. Program the autosampler to take samples at the desired time points; in general, samples are taken at 4, 8, 12, 24, and thereafter every 24 hours for the length of time desired
4. Prepare the dissolution solution from 3-4 L of DI water using a 0.1M sodium phosphate buffer adjusted to pH 7.2
5. Fill each test vessel with 500 mL of the dissolution solution
6. Turn on the circulating water bath on the dissolution apparatus, adjusting the temperature to 32° C. (+/−0.5° C.)
7. Prepare the samples to be tested
   a. Punch out 5 cm² circular samples from the patch to be tested. Samples are run in duplicate
   b. Clean the paddle with DI water followed by a suitable organic solvent and then let dry
   c. Remove the release liner from the patch and lightly attach one edge of the test sample to the inside of the stainless steel basket that is facing up
   d. Place the stainless steel basket with the sample into the test vessel. Ensure that the basket is laying inverted at the bottom of the vessel
8. Run the samples
   a. Once samples are placed in the vessels, lower the paddles and turn the paddles on. Set RPM to 70
   b. Start the autosampler
9. Once the dissolution samples have been collected, analyze the samples to determine drug release profile using HPLC
10. Lift the paddles and remove the vessels and sample basket. Clean all parts with soapy water followed by DI water and rinse with solvent

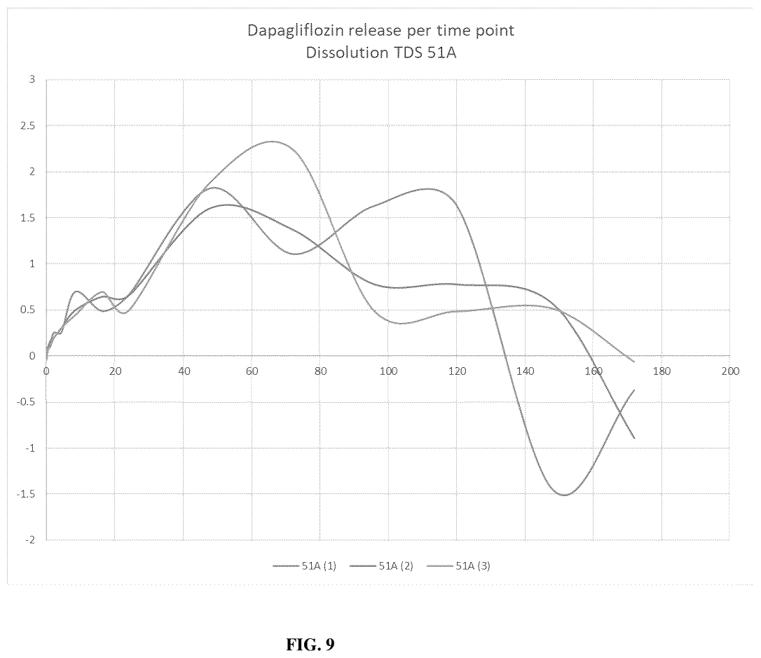

FIG. 9 provides the dapagliflozin release (mg) from dissolution studies of formulation 51A over 172 hours.

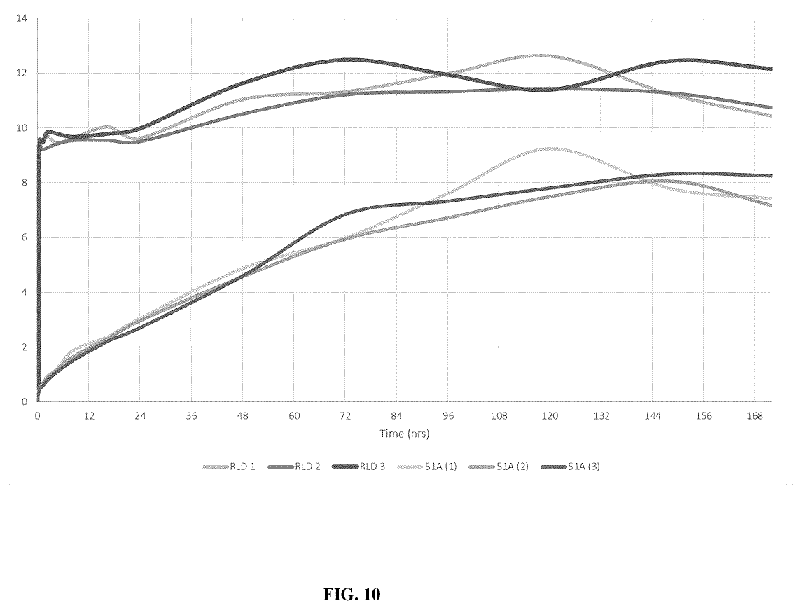

FIG. 10 compares the dapagliflozin release (mg) from dissolution studies of formulation 51A and the oral form of dapagliflozin over 172 hours.

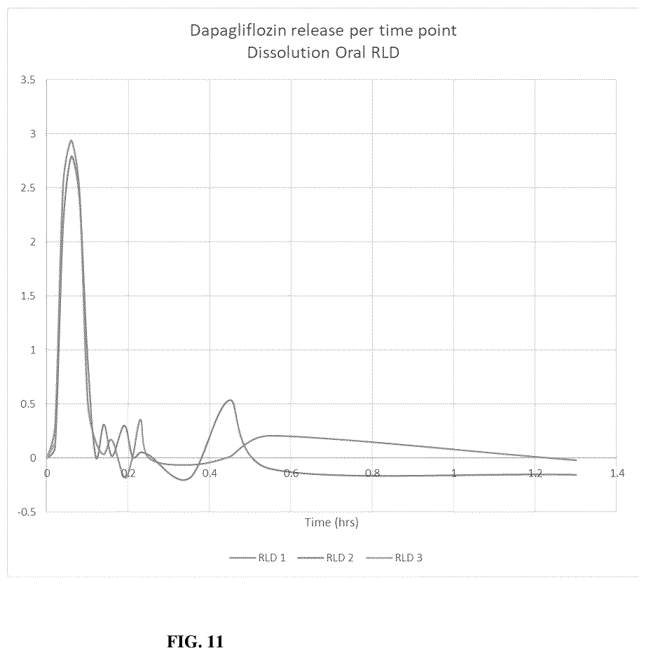

FIG. 11 provides the dapagliflozin release (mg) from dissolution studies of the oral form of dapagliflozin over 1.3 hours.

### Example 6: Stability of Dapagliflozin TDS Patches

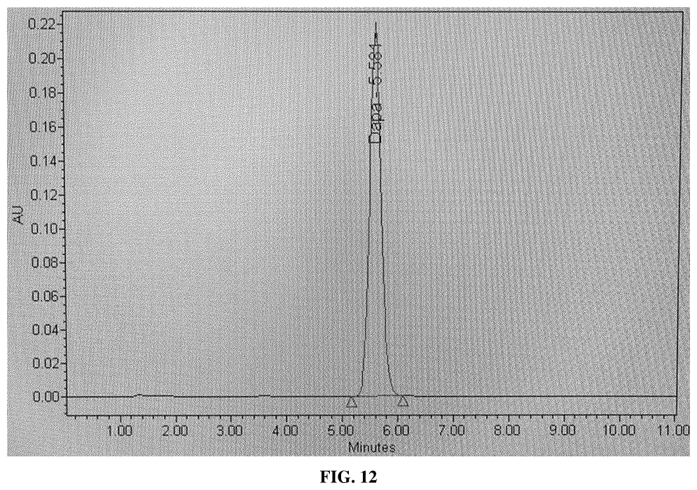

To determine the stability of the dapagliflozin TDS patches, unpouched 0.18 cm² samples were placed in a 37° C. oven and run on HPLC after 45 days. HPLC samples were prepared by extracting each patch with mobile phase for 7 days. Purity was determined based on percent area of the main dapagliflozin peak (representative HPLC trace shown in FIG. 12). TABLE 25 provides a summary of the percent purity of a blank sample, standard samples, and the dapagliflozin TDS patches.

TABLE 25

| | HPLC Retention Time | | | |
|---|---|---|---|---|
| | Main Dapa | Peak at | Peak | Peak |
| | Peak at | 1.6 | at 2.2 | at 2.6 |
| | 5.5 minutes | minutes | minutes | minutes |
| Sample | (% area) | (% area) | (% area) | (% area) |
| Standard 1 | 98.81 | 1.19 | 0 | 0 |
| Standard 2 | 98.98 | 1.02 | 0 | 0 |
| Standard 3 | 97.74 | 2.26 | 0 | 0 |
| Standard 4 | 99.14 | 0.86 | 0 | 0 |
| Standard 5 | 98.96 | 0.85 | 0 | 0.18 |
| Standard 6 | 99.24 | 0.76 | 0 | 0 |
| Standard 7 | 98.86 | 0.87 | 0 | 0 |
| Average | 98.82 | 1.12 | 0 | 0.03 |
| (Standards 1-7) | | | | |
| Blank | — | solvent front | 0 | 0 |
| 34C - 1 | 99.24 | 0.76 | 0 | 0 |
| 34C - 2 | 95.6 | 1.26 | 3.13 | 0 |
| 34B - 1 | 98.91 | 0.42 | 0 | 0 |
| 34B - 2 | 99.46 | 0.52 | 0 | 0 |
| 34D - 1 | 99.31 | 0.69 | 0 | 0 |
| 34D - 2 | 98.44 | 1.51 | 0 | 0 |

### Example 7: Wear Studies of Placebo TDS Patches

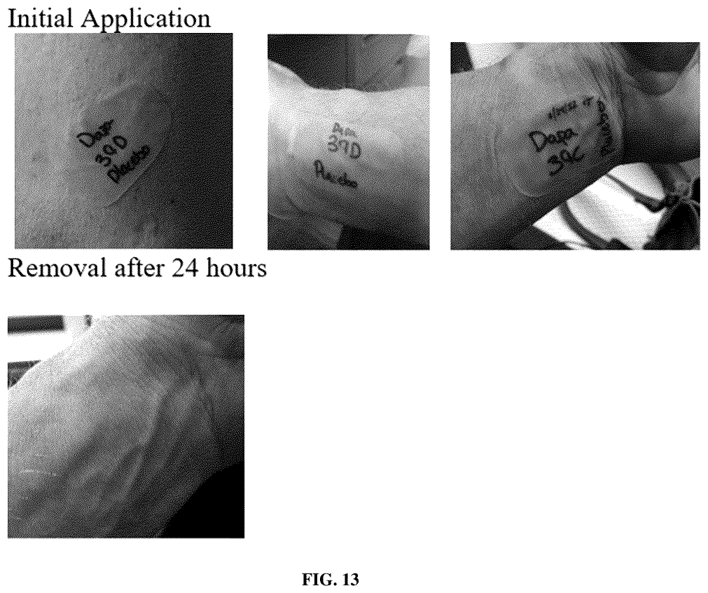

Placebo TDS patches were worn by three human subjects to determine initial wear and irritation. The placebo patches were 5 cm² and worn for 24 hours (including bathing cycles). Initial adhesion, patch removal, lifting, redness, and irritation were assessed on a scale of 1-5 (1=poor, 5=good) and summarized in TABLE 26. FIG. 13 shows photographs upon initial application and removal of the placebo patch after 24 hours.

TABLE 26

| | Initial | Patch | | Red- | Irri- |
|---|---|---|---|---|---|
| Subject | Adhesion | Removal | Lifting | ness | tation |
| Caucasian Male, Age 30 | 5 | 5 | 5 | None | None |
| Caucasian Female, Age | 5 | 5 | 5 | None | None |
| 25 | | | | | |
| Caucasian Male, Age 60 | 5 | 5 | 5 | None | None |

### Example 8: Cold Flow, Crystallization, and Freeze Thaw Evaluations of TDS Patches

### Study A: Cold Flow Testing

Cold flow was evaluated by visual observation of stickiness on the edges of the sample where they were punched out from the completed roll. Of the formulations tested (3B, 7A, 7C, 7D, 8A, 8B, 8C, 8D, 10A, 10C, 10D, 10E, 10F, 10G, 10H, 10K, 14A, 14B, 14C, 14D, 18A, 18B, 18D, 18E, 18F, 18G, 18I, 18J, 23A, 23B, 23C, 31B, 31C, 31D, 31E, 34A, 34B, 34C, 34D, 34E, 34F, 34G, 34H, 34I, 34J, 34K, 34L, 34M, 34N, 39B, 39C, 39D), none showed signs of stickiness on the edges.

### Study B: Crystallization Evaluation

Crystallization was evaluated by visual observation of snowflake-type crystals or needles and was observed in formulations that used Glycerol, Glycol, SDS, or Tween 20. Of the formulations tested (3B, 7A, 7C, 7D, 8A, 8B, 8C, 8D, 10A, 10C, 10D, 10E, 10F, 10G, 10H, 10K, 14A, 14B, 14C, 14D, 18A, 18B, 18D, 18E, 18F, 18G, 18I, 18J, 23A, 23B, 23C, 31B, 31C, 31D, 31E, 34A, 34B, 34C, 34D, 34E, 34F, 34G, 34H, 34I, 34J, 34K, 34L, 34M, 34N, 39B, 39C, 39D), crystals were only observed in in 8A, 8B, 10D, and 10F. These formulations contained glycerol, glycol, SDS, or Tween 20™ enhancers. These observations suggest that alcohol-containing compounds often induce crystallization.

### Study C: Freeze Thaw Studies

Samples (39B, 39C, and 39D) were inserted into a −80° C. freezer for four hours and then removed and allowed to reach room temperature (20° C.). Samples were then analyzed on HPLC for the presence of impurities. No impurities were detected and no crystal formation was observed by visual inspection.

### Example 9: Manufacturing of TDS Patches

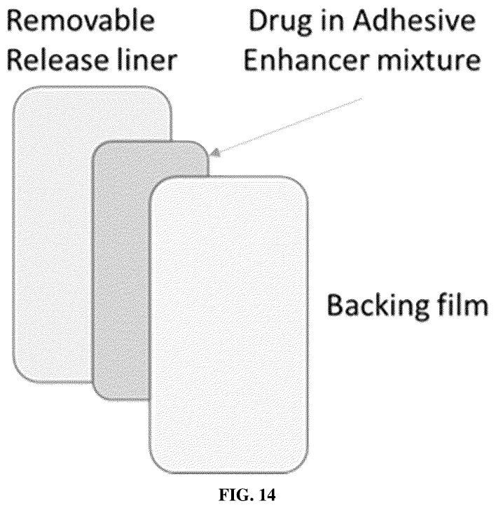

A monolith style patch was created having a removable release liner, a drug in adhesive and enhancer mixture, and a backing film (FIG. 14). The solution was coated on a 3M Scotchpak™ 9744 Release Liner Fluoropolymer Coated Polyester Film release liner at a thickness of 0.015 inches, and dried at 70° C. and 120° C. in a two-stage coater dryer. The product was laminated to a 3M CoTran™ 9720 Backing Polyethylene Monolayer Film. The manufacturing process included preparing the coating solution, drying, laminating, die cutting, and packaging.

The coating solution was prepared by adding solvent to the active pharmaceutical ingredient (dapagliflozin) and either stirring or rolling for a minimum of 20 minutes to achieve a complete solution. To this solution, the enhancer was added followed by the adhesive. The resulting mixture was stirred until a complete solution was obtained, typically 30 minutes. Fast mixing speeds were avoided to prevent undesirable aeration. Mixing was conducted at temperatures ranging between 60° F. and 80° F. The mixture can be held for up to 7 days with no change in final product. A 20 gram mixture makes enough solution for approximately 4 feet of laminate (40-60 patches).

A hand drawdown coater was used for small-scale manufacturing. Solutions for coating were applied using an adjustable knife over a glass plate onto the release liner. 10-20 mL of coating solution were used for a test film that was 12 inches long by 5-5.5 inches wide. The coating thickness was adjusted using built-in micrometers and verified by feeler gauges. The coatings were dried in a laboratory convection oven set at various temperatures or using the individual dryer zones of the pilot coater. The coatings were manually laminated and punched out with either a 5 cm² or 0.71 cm² cutter.

A general procedure for coating, drying, and laminating a small-scale formulation is as follows:
1. Using a knife over plate or glass hand drawn apparatus, set the knife to the desired coat thickness over the 9744 release liner. Using the built-in micrometer or feeler gauge, verify the coat thickness on both sides of the knife. The release liner is 10-16 inches long for the hand drawdown with treated side facing up. This length is confirmed using a permanent marker to make a small line on either side. The side where the marker does not stick is the treated side used for coating.
2. Coat thickness for the solution is set to 0.015 inches (38 mm)
3. Set the dam width to the desired coat width (for a nominal 6-inch film width, set to a 4-5 inch coating width)
4. With the knife set to the proper height and the dam set to the proper coating width, place 5-20 mL of solution in front of the knife and immediately start drawing down the solution (takes about 10-20 seconds over a 10-16 inch length of release liner)
5. The coating is completed. Clean up using acetone or ethyl acetate once the films are in the dryer
6. Place the coated film into the Stage 1 convection oven set at 75-80° C. Dry the film for 1-2 minutes (+/−0.5 minutes) at 75-80° C. After Stage 1 is complete, move the coated film to the Stage 2 dryer set at 115-120° C. Dry the film for 2-3 minutes (+/−0.5 minutes).
7. Remove the dried film from the oven and let cool to room temperature (about 1-2 minutes)
8. Using a manual laminator, Nip the dried film to the 9720 backing film using moderate to light pressure (about 1-10 grams of force, depending on the lab equipment, or 50-75% of the total release liner, coating, and backing film thickness)
9. Store the final product at room temperature away from light

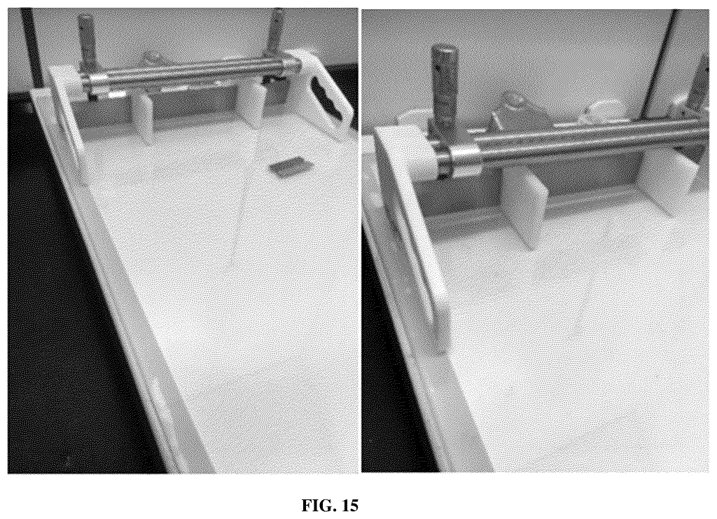

FIG. 15 provides a top view of the hand drawdown coater.

Formulations are scaled up using the pilot coating machine, which uses the same mechanism for adjusting coat thickness as the hand drawdown coater. The drying occurs in two stages, each with variable speed inlet and exhaust fans. The coated film is nipped in the machine and rewound onto a spool. Coatings can be 20 inches to 50 feet in length with widths from 1 to 5.5 inches wide.

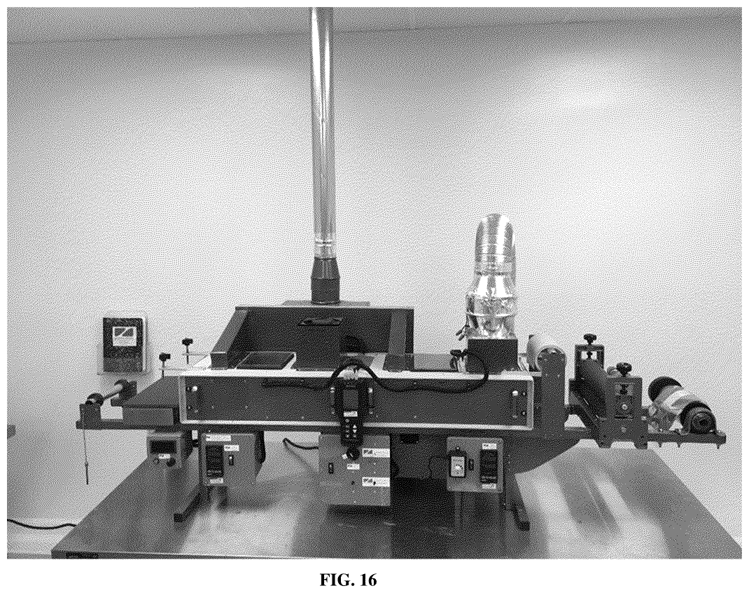

FIG. 16 provides a side view of the pilot coating machine.

Placebo patches were made from a commercial coater manufactured by HED to validate the pilot coater comparability to a commercial scale coater. The pilot coater functions and produces coatings with the same content uniformity, coat thickness, drying, tack, and shear as the commercial coater.

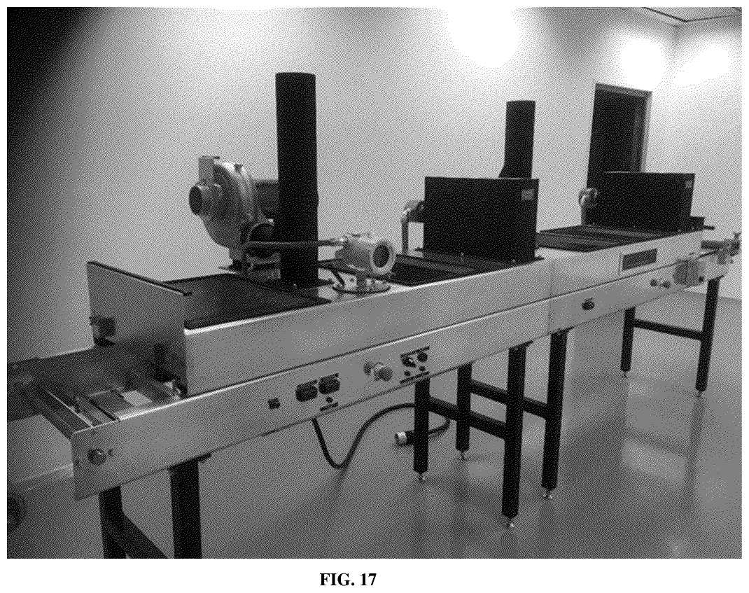

FIG. 17 shows a side view of the commercial coating machine.

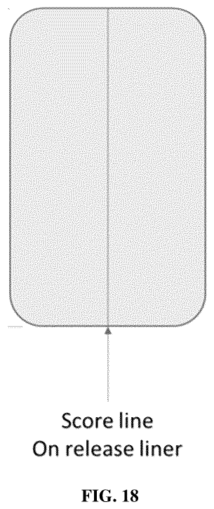

A general procedure for die cutting is as follows:
1. The patch is cut out in a rectangle with rounded edges
2. The product is die cut or cut using a computer-controlled knife
3. A score is cut into the release liner through the middle of the patch edge to edge (FIG. 18)
4. The cut material is collected and stored at room temperature away from light

A general procedure for packaging is as follows:
1. The pouch is constructed of aluminum-faced packaging material sealed on all sides
2. The patch is inserted into the packaging material
3. All four sides are heat sealed with temperatures about 250-350° F. with a 1 second dwell time
4. The package is embossed with the lot number and scored. The product machine, product name, and other information printed on the package or pouching material can be pre-printed
5. The pouched material is inserted into the final shelf carton. The product can be stored at room temperature

### Coating Solution—Controls

1. All ingredients are weighed and verified by a second person
2. The product is visually inspected for the presence of foreign particulates or undissolved material
3. The coating solution is assayed (weight/weight %) to ensure the correct amount of active ingredient has been added

### Coating, Drying, Laminating—Controls

1. The release liner film is checked to ensure the treated side faces the coat head
2. The coat head is adjusted using installed micrometers. The coat head height is verified using a metal feeler gauge adjusted to ensure both the sides and middle of the knife is set to the proper height. The dam width measured to ensure the coat width is at least 0.75 inches from the film edge
3. For commercial scale equipment, samples are taken from the laminated roll prior to the rewind station. Samples are punched out into the desired size and immediately weighed and have the thickness confirmed.
   a. Samples from the left, middle, and right, beginning, middle, and end are punched from the completed roll
   b. 1-inch samples are cut from the film and weighed for total weight. 1-inch samples of the release liner and backing film are also be taken and weighed. The total coat weight is calculated: (Sample Weight)−(Release Liner)−(Backing Film)=Coat Weight
   c. 1-inch samples are tested for coating thickness using a micrometer. The samples measured are within the coat range. The total coat thickness is calculated: (Sample Thickness)−(Release Liner)−(Backing Film)=Coat Thickness
   d. 1-inch samples are observed for bubbles, streaks, or other physical abnormalities prior to testing

### Die Cutting—Controls

1. Once die cutting has begun, random samples are pulled to confirm product width (measured using a ruler or micrometer)
2. Random samples from the beginning, middle, and end of the cycle are checked for score. Visual observations are made to confirm the release liner was not cut through

### Packaging—Controls

1. Samples are collected every 15 minutes and checked for seal integrity using a burst machine
2. Pouches are open and placement of the patch within the pouch is observed to ensure that the patch has not moved into the sealed area

### Example 10: Pharmacokinetic (PK) Studies Performed in Animal and Human Subjects

### Pharmacokinetic Study—Rats

A pharmacokinetic study is performed with four dapagliflozin TDS patches in male SD rats. The right or left-dorsal region of the rat is shaved with a razor blade and soap or shaving foam, rinsed with water, and dried prior to applying each patch. Each patch is applied for 7 days. If the patch comes off, gauze is put over the patch and tape is placed on the ends of the gauze. Rats have free access to food and water. The study occurs in four phases listed in TABLE 27.

TABLE 27

| | | | Patch | | |
|---|---|---|---|---|---|
| | Patch | | Dura- | Sampling | |
| Phase | # | Patch Region | tion | Times (hours) | Observations |
| 1 | 1 | right-dorsal | 7 days | 1, 4, 8, 12, 24, | Record |
| | | | | 48, 72, 96, | bodyweight |
| | | | | 120, 144, and | and clinical |
| | | | | 168 | symptoms |
| 2 | 2 | left-dorsal | 7 days | 1, 4, 8, 12, 24, | once daily, |
| | | | | 48, 72, 96, | including skin |
| | | | | 120, 144, and | irritation and |
| | | | | 168 | patch adhesion |
| 3 | 3 | same area on | 7 days | 1, 4, 8, 12, 24, | |
| | | right-dorsal | | 48, 72, 96, | |
| | | side where | | 120, 144, and | |
| | | Patch 1 was | | 168 | |
| | | applied | | | |
| 4 | 4 | same area on | 7 days | 1, 4, 8, 12, 24, | |
| | | left-dorsal side | | 48, 72, 96, | |
| | | where Patch 2 | | 120, 144, and | |
| | | was applied | | 168 | |

A 3-week washout period occurs after the last timepoint in Phases 1, 2, and 3. Additional plasma samples are collected on the 7^{th} and 14^{th} day from the beginning of the washout period after the last timepoint in Phase 1 to determine whether a 3-week washout is sufficient. Bioanalytical methods are used to analyze the plasma samples from Phases 1-4.

### Pharmacokinetic Study—Humans

An open label study is conducted in healthy humans to determine the single dose pharmacokinetics of dapagliflozin TDS patches. Subjects are admitted to the clinical unit the morning prior to product administration and remain on site until 72 hours post dose blood sample has been taken. Subjects are selected from healthy male and women of non-childbearing potential aged 18 to 55 years with a body mass index of 18-32 kg/m² and a good state of health as determined by a comprehensive clinical assessment.

Safety assessments are performed on subjects at screening, admission, and post dose. Urinalysis is performed at screening admission and 72 hours post dose. Vital signs (blood pressure, pulse, temperature, and respiratory) are performed at screening, admission, and at 4 time points post dose, and performed daily on non-dosing days during clinical confinement. Electrocardiograms are performed at screening, admission, and at 4 time points post dose. Patch adhesion and local tolerability are assessed regularly during the clinical duration. Physical examination is performed at screening. A symptom driven examination is performed at 24 hours post dose. Drugs of abuse, alcohol, and cotinine urine tests are performed at screening and each admission. Tests for Hepatitis B, C, and HIV are performed at screening. A follow-up phone call occurs 3-7 days post final dose to ensure the ongoing wellbeing of the subjects.

Blood samples from the subjects are taken at the following times: pre-dose and 0.5, 1, 1.5, 2, 3, 4, 6, 8, 10, 12, 16, 20, 24, 30, 36, 48, and 72 hours after dosing. Plasma samples are analyzed using software to obtain the following parameters: lag time (Tlag), time of occurrence of Cmax (Tmax), maximum observed concentration (Cmax), area under the curve from pre-dose to 24 hours (AUC (0-24)), area under the curve from pre-dose to the last sample (AUC (0-last)), area under the curve extrapolated to infinite time (AUC (0-inf)), % AUC extrapolated beyond last measured time point, terminal rate constant (λz), half-life (T½), oral clearance (Cl/F), and volume of distribution (Vz/F).

### Example 11: Transdermal Patch Specifications

A transdermal patch has the following specifications:
Appearance—rectangular patch, tan color on top, bright yellow color on bottom
Dimension—27.5 cm²
Patch thickness—0.2125 inches+/−13%
Coat thickness—0.015 inches (38 millimeters) as is
Patch weight—0.121 grams+/−5% (5 cm² sample size)
Coat weight—0.0181 grams+/−3% (5 cm² sample size)
Tack adhesion—no less than 17 seconds at a minimum of 8 grams pressure
Shear adhesion—no less than 269 grams pressure
90 Degree Peel—100-250 grams force
180 Degree Peel—100-300 grams force
Dapagliflozin—6.7 mg/5 cm² patch
% Dapagliflozin “solids” basis—34-37%
Assay—98.0-102.0% as Dapagliflozin
Impurity profile—Total impurity <0.1%, major impurity <0.1%
Residual solvent—NMT 500 ppm ethyl acetate, or NMT 500 ppm ethanol or methanol

### Example 12: Major Indications of the Transdermal Patch

The active ingredients described herein—including SGLT2 inhibitors, alpha-glucosidase inhibitors, DPP-4 inhibitors, GLP-1 agonists, meglitinides, metformin, sulfonylureas, thiazolidinediones, amylin analogues, and insulin—are collectively called antidiabetic agents or more specifically, antihyperglycemic agents. Their primary indication is the management of diabetes mellitus (mainly type 2, though some are also approved for type 1 and specific sub-populations), but many have additional, increasingly broad indications.

### SGLT2 Inhibitors—Indications

Type 2 Diabetes Mellitus (T2DM): All SGLT2 inhibitors (canagliflozin, dapagliflozin, empagliflozin, ertugliflozin, ipragliflozin, luseogliflozin, tofogliflozin, and their solvates/hydrates) are approved as adjuncts to diet and exercise to improve glycemic control in adults with T2DM.
Heart Failure: Dapagliflozin and empagliflozin are indicated to reduce the risk of hospitalization for heart failure in adults, including those with reduced or preserved ejection fraction, regardless of diabetic status.
Chronic Kidney Disease (CKD): Dapagliflozin is indicated to reduce the risk of sustained eGFR decline, end-stage kidney disease, cardiovascular death, and hospitalization for heart failure in patients with CKD (with or without diabetes).
Type 1 Diabetes (select agents/countries): Some SGLT2 inhibitors (e.g., dapagliflozin, not FDA-approved for T1DM in the US but approved elsewhere) may be used as adjunct therapy in T1DM for selected patients under specific restrictions.

### Other Antidiabetic Agents—Indications

Alpha-glucosidase inhibitors (acarbose, miglitol): Used to treat T2DM by delaying carbohydrate digestion and absorption.
Amylin analogue (pramlintide): Used to treat T1DM and T2DM in patients using mealtime insulin to improve glycemic control.
DPP-4 inhibitors (alogliptin, linagliptin, saxagliptin, sitagliptin): Indicated for T2DM management, usually as adjuncts to diet and exercise.
GLP-1 agonists/incretin mimetics (albiglutide, dulaglutide, exenatide, liraglutide, lixisenatide): Indicated for T2DM; some (liraglutide, dulaglutide, semaglutide—not on the list) also carry indications to reduce the risk of major adverse cardiovascular events (MACE) in T2DM with CV risk.
Meglitinides (nateglinide, repaglinide): Indicated for T2DM, particularly for controlling postprandial hyperglycemia.
Metformin: First-line antihyperglycemic for T2DM; also used off-label for prediabetes and conditions with insulin resistance.
Sulfonylureas (chlorpropamide, glimepiride, glipizide, glyburide, tolazamide, tolbutamide): Indicated for T2DM, often used when metformin is insufficient or contraindicated.
Thiazolidinediones (rosiglitazone, pioglitazone): Indicated for T2DM; pioglitazone may also be used in combination with insulin.
Insulin: Indicated for T1DM and advanced or uncontrolled T2DM. Can be significant for T1DM and used in various forms (rapid, short, intermediate, long-acting) for both types of diabetes and emergency hyperglycemia.

All these active ingredients can be grouped under the term “antidiabetic agents,” with more specific subcategories such as:
Oral hypoglycemic agents (non-insulin oral drugs for T2DM)
Non-insulin antidiabetic agents (all non-insulin diabetes drugs)
Antihyperglycemic drugs (agents that lower elevated blood glucose levels)

They are most broadly categorized as active ingredients for the treatment of diabetes.

Summary Table: Major Indications of the Transdermal Patch

| Drug Class/Example | Main Indications | Select Additional Indications |
|---|---|---|
| SGLT2 inhibitors (e.g., dapagliflozin) | T2DM | Heart failure, CKD, select T1DM (outside US) |
| Alpha-glucosidase inhibitors (acarbose, etc.) | T2DM | — |
| Amylin analogue (pramlintide) | T1DM, T2DM (with insulin) | — |
| DPP-4 inhibitors (e.g., sitagliptin) | T2DM | — |
| GLP-1 agonists (e.g., liraglutide) | T2DM | CV risk reduction, weight management |
| | | (some agents) |
| Meglitinides (repaglinide, nateglinide) | T2DM | — |
| Metformin | T2DM (first-line), PCOS (off-label) | Prediabetes |
| Sulfonylureas (glimepiride, etc.) | T2DM | — |
| Thiazolidinediones (pioglitazone, etc.) | T2DM | — |
| Insulin | T1DM, T2DM (advanced/uncontrolled), | Gestational diabetes, DKA/HHS |
| | emergencies | |

1. A transdermal patch comprising:
at least one SGLT2 inhibitor or antidiabetic agent;
an adhesive layer containing an acrylic adhesive in which the SGLT2 inhibitor and/or or antidiabetic agent is incorporated;
optionally, one or more permeation enhancers in the adhesive layer, to facilitate delivery of the SGLT2 inhibitor and/or or antidiabetic agent through the skin;
a removeable release liner, positioned on one surface of the adhesive layer, configured to be removed prior to application; and
a backing film on the opposed surface of the adhesive layer, impermeable or occlusive to protect the patch and control drug release direction;
wherein the adhesive layer is positioned between the removable release liner and the backing film, such that upon removal of the liner and placement on a skin surface, drug permeation is directed outward from the adhesive-contacting layer toward the skin.

2. The transdermal patch of claim 1, wherein the SGLT2 inhibitor comprises canagliflozin, dapagliflozin, empagliflozin, ertugliflozin, ipragliflozin, luseogliflozin, tofogliflozin, a solvate or hydrate thereof.

3. The transdermal patch of claim 1, wherein the antidiabetic agent inhibitor comprises alpha-glucosidase inhibitors (acarbose, miglitol); amylin analogue (pramlintide); dipeptidyl peptidase 4 (DPP-4) inhibitor (alogliptan, linagliptin, saxagliptin, sitagliptin); incretin mimetics/GLP-1 agonist (albiglutide, dulaglutide, exenatide, liraglutide, lixisenatide); meglitinide (nateglinide, repaglinide), metformin (biguanide), sulfonylureas (chlorpropamide, glimepiride, glipizide, glyburide, tolazamide, tolbutamide), thiazolidinedione (rosiglitazone, pioglitazone), and insulin.

4. The transdermal patch of claim 1, wherein the adhesive layer contains an acrylic adhesive functionalized with the crosslinking groups carboxyl (—COOH) and/or hydroxyl (—OH)).

5. The transdermal patch of claim 1, wherein the permeation enhancer comprises organic acids, nonionic surfactants, or fatty acid derivatives.

6. The transdermal patch of claim 1, wherein the permeation enhancer comprises the organic acid levulinic acid or lactic acid; the nonionic surfactant polysorbate 80; or fatty acid derivatives.

7. The transdermal patch of claim 1, wherein the removeable release liner comprises a polyester film or fluoropolymer-coated polyester.

8. The transdermal patch of claim 1, wherein the backing film comprises polyethylene monolayer film, polyester, or laminate of polyethylene and aluminum vapor-coated polyester.

9. The transdermal patch of claim 1, wherein the SGLT2 inhibitor is physically and/or chemically integrated into the adhesive matrix, uniformly dispersed or dissolved within the acrylic adhesive.

10. The transdermal patch of claim 1, wherein the SGLT2 inhibitor is further mixed solvent.

11. The transdermal patch of claim 1, wherein the SGLT2 inhibitor is further mixed the solvent methanol, ethyl acetate, or combination thereof.

12. The transdermal patch of claim 1, wherein the adhesive layer further comprises one or more additional excipients, stabilizers, carriers, diluents, dispersants, thickeners, or agents for manufacturability or stability.

13. The transdermal patch of claim 1, having a surface area of from about 1 square centimeter to about 100 square centimeters.

14. The transdermal patch of claim 1, having a thickness from about 0.1 millimeter to about 10 millimeters.

15. The transdermal patch of claim 1, having a mass of from about 10 milligrams to about 10 grams.

16. The transdermal patch of claim 1, wherein the adhesive layer comprises a single, monolithic drug-in-adhesive layer and contains no more than one drug-in-adhesive layer, thereby increasing manufacturing reproducibility and patch consistency.

17. The transdermal patch of claim 1, wherein the patch is configured to provide sustained release of the SGLT2 inhibitor or antidiabetic agent over a period of at least 7 days upon application to the skin.

18. The transdermal patch of claim 1, wherein application to human skin for up to 7 days delivers at least 50% by weight of the total SGLT2 inhibitor or antidiabetic agent content measured by residual analysis post-removal.

19. The transdermal patch of claim 1, wherein the patch demonstrates effective transdermal delivery of SGLT2 inhibitors, as confirmed by skin permeation testing using human or animal skin, yielding unexpectedly efficient delivery despite the moderate polarity and molecular weight of the SGLT2 inhibitor.

20. The transdermal patch of claim 1, wherein the SGLT2 inhibitor is released from the patch in a controlled, sustained manner over 7 days in an acidic environment, wherein the total release after 7 days is less than the total release from an orally administered form after 12 hours, resulting in reduction of peak-related systemic side effects.

21. The transdermal patch of claim 1, wherein the patch is configured to exhibit a tack value of at least 5 grams, measured by force required to separate the patch from a stainless-steel cylinder.

22. The transdermal patch of claim 1, wherein the patch exhibits a 90° peel adhesion of at least 50 grams, as measured by force required to pull the patch from a steel plate at a 90° angle.

23. The transdermal patch of claim 1, wherein the patch exhibits a 180° peel adhesion of at least 50 grams, as measured by force required to pull the patch from a steel plate at a 180° angle.

24. The transdermal patch of claim 1, wherein the patch does not exhibit significant cold flow or edge tack under ambient storage, as determined by visual inspection after die-cutting and storage.

25. The transdermal patch of claim 1, wherein the patch is free of visible crystal formation or phase separation during extended storage or after freeze-thaw cycling, when formulated without glycerol, glycol, sodium dodecyl sulfate, or Tween 20.

26. The transdermal patch of claim 1, wherein the patch maintains greater than 95% purity of the SGLT2 inhibitor after at least 45 days storage at 37° C., as measured by HPLC, with no major impurities detected.

27. The transdermal patch of claim 1, wherein the release liner and backing films are each made from commercially available medical-grade films, enabling regulatory approval and scalable manufacturing.

28. The transdermal patch of claim 1, wherein the patch is packaged in a sealed aluminum pouch, the pouch being heat-sealed and configured to protect the patch from light and moisture during storage and shipping.

29. The transdermal patch of claim 1, wherein the patch is designed to exhibit robust adhesion to skin, minimal lifting, and low irritation or redness after 24 hours of continuous wear, as demonstrated in human wear studies.

30. The transdermal patch of claim 1, wherein residual solvent levels in the patch are less than 500 ppm for each of methanol, ethanol, and ethyl acetate, upon release for commercial distribution.
